(19) United States
(12) Patent Application Publication
(10) Pub. No.: US 2008/0279794 A1
(43) Pub. Date: Nov. 13, 2008

(54) Use of halogenated hydroxydiphenyl ether compounds for the treatment of the skin

(76) Inventors: Werner Baschong, Basel (CH); Marcel Schnyder, Basel (CH)

Correspondence Address: JoAnn Villamizar;Ciba Corporation/Patent Department, 540 White Plains Road, P.O. Box 2005, Tarrytown, NY, 10591, US

(21) Appl. No.: 12/214,998

(22) Filed: Jun. 24, 2008

Related U.S. Application Data
(62) Division of application No. 10/564,712, filed on Jan. 12, 2006.

(30) Foreign Application Priority Data
Jul. 16, 2003 (EP) 03405542.6

(51) Int. Cl. A61K 8/49; A61Q 19/02; A61Q 17/04

(52) U.S. Cl. 424/59; 510/131

(57) ABSTRACT

The use of
(a) hydroxydiphenyl ether compounds of formula

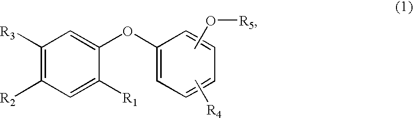

wherein
   R₁, R₂ and R₃ are each independently of the others hydrogen; hydroxy; C₁-C₂₀alkyl; hydroxy-substituted C₁-C₂₀alkyl; C₅-C₇cycloalkyl; C₁-C₂₀alkoxy; C₁-C₆alkylcarbonyl; phenyl; or phenyl-C₁-C₃alkyl;
   R₄ is hydrogen, C₁-C₂₀alkyl; hydroxy-substituted C₁-C₂₀alkyl; C₅-C₇cycloalkyl; hydroxy; formyl; acetonyl; allyl; carboxy; carboxy-C₁-C₃alkyl; carboxyallyl; C₂-C₂₀alkenyl; C₁-C₆alkylcarbonyl; C₁-C₃alkylcarbonyl-C₁-C₃alkyl; phenyl; or phenyl-C₁-C₃alkyl; and
   R₅ is hydrogen; C₁-C₂₀alkoxy; or C₁-C₆alkylcarbonyl;
      as melanogenesis inhibitors and for lightening the skin.

This application is a divisional of application Ser. No. 10/564,712, filed Jan. 12, 2006, pending, the disclosure of which is hereby incorporated by reference.

The present invention relates to the use of halogenated hydroxydiphenyl ether compounds for the treatment of the skin, especially for lightening the skin and as a melanogenesis inhibitor.

There is intense interest in preparations which not only do not alter the colour of the skin after exposure to sunlight but are also capable of imparting a lighter appearance to the skin by reducing the production of the skin pigment melanin or by lightening its colour. In Asian countries in particular, a light-coloured skin tone, that is to say skin lightening or protection against tanning, is desirable.

The problem underlying the present invention is therefore to find compositions that are able to prevent tanning of the skin and at the same time lighten the skin.

Surprisingly, it has now been found that certain halogenated hydroxydiphenyl ether compounds are able to solve this problem.

The present invention therefore relates to the use of

(a) halogenated hydroxydiphenyl ether compounds of formula

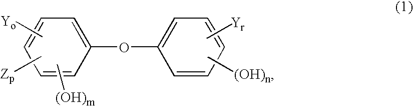

wherein
Y is chlorine or bromine,
Z is SO₂H, NO₂; or C₁-C₄alkyl;
m is 0 or 1;
n is 1 or 2;
r is from o to 3;
o is from 1 to 3; and
p is 0, 1 or 2;
as melanogenesis inhibitors and for lightening the skin.

C₁-C₂₀Alkyl is a straight-chain or branched alkyl group, e.g. methyl, ethyl, n-propyl, isopropyl, n-butyl, sec-butyl, tert-butyl, amyl, isoamyl or tert-amyl, hexyl, heptyl, octyl, isooctyl, nonyl, decyl, undecyl, dodecyl, tetradecyl, pentadecyl, hexadecyl, heptadecyl or octadecyl.

C₁-C₂₀Alkoxy is a straight-chain or branched alkoxy group, e.g. methoxy, ethoxy, n-propoxy, isopropoxy, n-butoxy, sec-butoxy, tert-butoxy, amyloxy, isoamyloxy or tert-amyloxy, hexyloxy, heptyloxy, octyloxy, isooctyloxy, nonyloxy, decyloxy, undecyloxy, dodecyloxy, tetradecyloxy, pentadecyloxy, hexadecyloxy, heptadecyloxy or octadecyloxy.

C₂-C₁₈Alkenyl is, for example, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₁-C₆Alkylcarbonyl is a straight-chain or branched carbonyl group with an alkyl radical having from one to six carbon atoms, e.g. acetyl, propionyl, butyryl, isobutyryl, valeryl, isovaleryl, pivalolyl and the like.

Suitable hydroxy-substituted C₁-C₂₀alkyl groups are, for example, hydroxymethyl, hydroxyethyl, hydroxypropyl, hydroxybutyl, hydroxypentyl, hydroxyhexyl, hydroxyheptyl, hydroxyoctyl, hydroxynonyl, hydroxydecyl and the like.

It is preferable to use halogenated hydroxydiphenyl ethers of formula (1) wherein

m is 0; or 1;
n is 1; or 2;
o is from 1 to 3;
p is 0; or 1; and
r is 1 or 2.

Special preference is given to compounds of formula

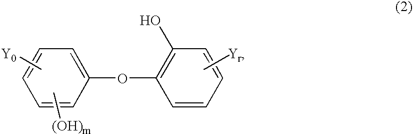

wherein
m is 0; or 1;
o is from 1 to 3; and
r is 1 or 2.

Preferably, in formula (2),

m is o; and
o and r are as defined in claim 3.

Special preference is given to hydroxydiphenyl ethers of formula (2) wherein

o is 1 or 2; and
r is 1.

According to the invention, very special preference is given to the use of compounds of formulae

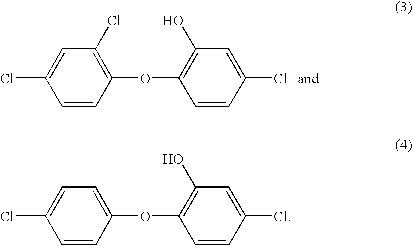

The halogenated hydroxydiphenyl ether compounds of formula (1) used according to the invention can also be used for the simultaneous antimicrobial treatment of organic surfaces.

The hydroxydiphenyl ether compounds of formula (1) used according to the invention exhibit a pronounced antimicrobial action, especially against pathogenic gram-positive and gram-negative bacteria and also against bacteria of skin flora, and additionally against yeasts and moulds. They are therefore especially suitable for the disinfection, deodorisation and the general and antimicrobial treatment of the skin and mucosa and also of integumentary appendages (hair), more especially for the disinfection of the hands and of wounds.

They are therefore suitable as antimicrobial active ingredients and preservatives in personal care preparations, for example shampoos, bath additives, hair-care products, liquid and solid soaps (based on synthetic surfactants and salts of saturated and/or unsaturated fatty acids), lotions and creams, deodorants, other aqueous or alcoholic solutions, e.g. cleansing solutions for the skin, moist cleansing cloths, oils or powders.

In addition to the halogenated hydroxydiphenyl ether compounds of component (a), it is additionally possible according to the invention to use further substances having skin-lightening properties (component (b)).

As component (b) it is possible to use, for example, the following substances or classes of substance:
1. pyrone derivatives, corresponding to formula

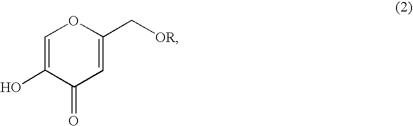

wherein
   R is hydrogen (=kojic acid; 5-hydroxy-2-hydroxymethyl-4H-pyran-4-one); or the radical of formula

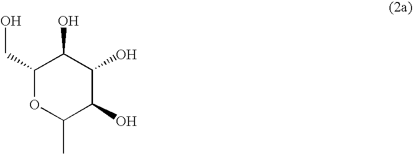

and derivatives thereof, e.g. kojic acid glucosides;
2. hydroquinone, including in the form of glycosides, and hydroquinone derivatives in the form of glycosides, e.g. 4-hydroxyphenyl-D-glucopyranoside (=α-, or β-arbutin), corresponding to formula

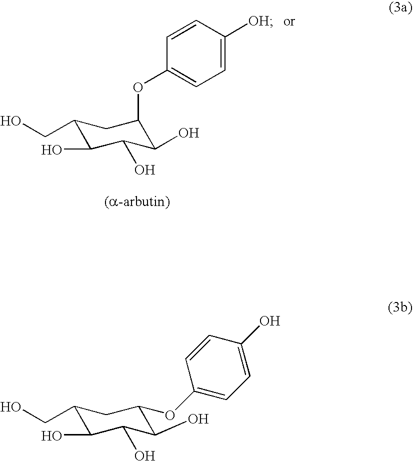

(β-arbutin); 4-methoxyphenethylmethyl ether D-glucopyranoside; 1,5,9,13-tetramethyl-4,8,12-tetradecatrienyl (9Cl); 5,9,13-pentadecatrien-2-ol, 6,10,14-trimethyl-(9Cl); 1,5,9,13-tetramethyltetradecyl D-glucopyranoside;
3. resorcinol derivatives such as glabridin (1,3-benzenediol, 4-[(3R)-3,4-dihydro-8,8-dimethyl-2H,8H-benzo[1,2-b:3,4-b′]dipyran-3-yl]-) or 4-butylresorcinol (=rucinol); 2,4-dihydroxybenzophenones and isomeric benzophenones;
4. glycine, L-α-glutamyl-L-cysteinyl-(=glutathione); acetylcysteine; oligopeptides;
5. alkyldicarboxylic acids, such as azelaic acid (nonanedicarboxylic acid) and mono- and di-esters thereof;
6. 1,2-dihydroxyphenyl derivatives, e.g. 4-(3,4-dihydroxyphenyl)butan-2-ol; 4-hydroxy-3-methoxybenzylacetone (=gingerone); 4H-1-benzopyran-4-one, 2-(3,4-dihydroxyphenyl)-3,5,7-trihydroxy-(=quercitin), corresponding to formula

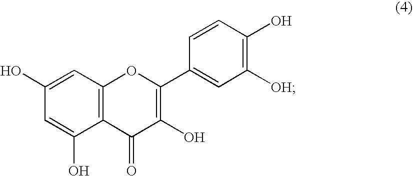

7. urea, (2,5-dioxo-4-imidazolidinyl)-(=allantoin), corresponding to formula

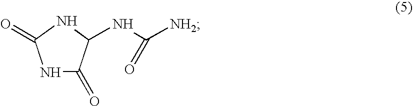

8. furanones, such as 3-hydroxy-4,5-dimethyl-2(5H)-furanone; 3-hydroxy-4-methyl-5-ethyl-2(5H)-furanone;
9. phenylacetaldehydes;
10. benzaldehydes, e.g. 4-hydroxybenzaldehyde and 3-methylbenzaldehyde;
11. 4-methoxycinnamaldehydes;
12. isomeric decenoic acid (C₁₀H₁₈O₂);
13. ascorbic acid and derivatives, for example 6-acylascorbic acid 2-glucoside; sulfates, stearates or phosphates of ascorbic acid;
14. salicylic acid derivatives, such as 6-[(8Z)-8-pentadecenyl]-salicylic acid; (anacardic acid monoene) and 6-[(8Z,11Z)-8,11,14-pentadecatrienyl]-salicylic acid (anacardic acid trienes);
15. phenolic substances, such as 3-[8(Z)-pentadecenyl]phenol or curcumin phenolic substances, e.g. curcumin;
16. benzo[b]pyran derivatives, such as [1]benzopyrano[5,4,3-cde][1]benzopyran-5,10-dione,
2,3,7,8-tetrahydroxy-(7Cl,8Cl,9Cl) (=ellagic acid); 2′-hydroxy-2,4,4,7,4′-penta-methylflavan; 2′-flavanol, 2,4,4,4′,7-pentamethyl-, acetate; 2-(3,4-dihydro-2,4,4,7-tetramethyl-2H-1-benzopyran-2-yl)-5-methylphenyl and 8-α-glycopyranosyl-7-hydroxy-5-methyl-2-(2-oxopropyl)-4H-1-benzopyran-4-one (aloesin), corresponding to formula

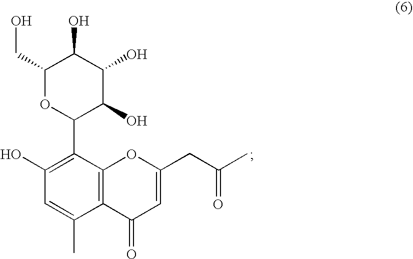

17. bornyl and cinnamate derivatives, such as 2-propenoic acid, 3-(4-hydroxyphenyl)-, 1,7,7-trimethylbicyclo[2.2.1]hept-2-yl ester, endo-; 2-propenoic acid, 3-(4-methoxyphenyl)-, 1,7,7-trimethylbicyclo[2.2.1]hept-2-yl ester, endo-; 2-propenoic acid, 3-(4-hydroxyphenyl)-, 1-methyl-3-(2,2,6-trimethylcyclohexyl)propyl ester; 2-propenoic acid, 3-phenyl-, 1-methyl-3-(2,2,6-trimethylcyclohexyl)propyl ester; 2-propenoic acid, 3-[4-(α-D-glucopyranosyloxy)phenyl]-, (1R,2S,4R)-1,7,7-trimethylbicyclo[2.2.1]hept-2-yl ester;
18. azulene and derivatives thereof, for example guajazulene or vetivazulene, and also guaiol;
19. cell messenger substances, such as cytokines; prostaglandins and peptide growth factors;
20. α-hydroxy-carboxylic acids, for example α-hydroxypropionic acid (lactic acid) and also citric acid and aconitic acid;
21. compound of formula

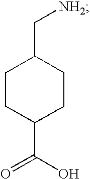

22. compound of formula

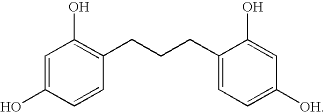

Preferred fluorescent whiteners suitable for use according to the invention correspond to formulae

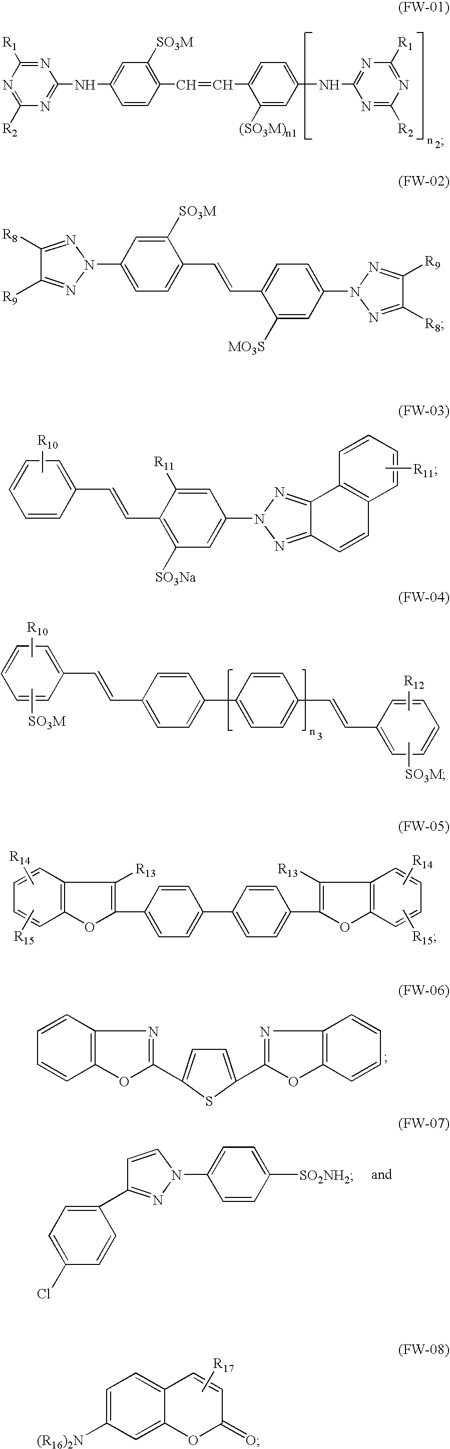

wherein
R₁ is a radical of formula

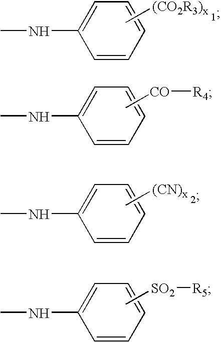

or Hal;
R₃ is M; an unsubstituted or substituted alkyl or aryl;
R₄ is hydrogen; an unsubstituted or substituted alkyl or aryl; or —NR₆R₇, wherein R₆ and R₇ are each independently of the other hydrogen; unsubstituted or substituted alkyl; or aryl; or R₆ and R₇ together with the nitrogen atom linking them form a heterocyclic radical, especially a morpholino or piperidino radical;
R₅ is hydrogen; an unsubstituted or substituted alkyl or aryl; or a radical of formula (1a) —(CH₂)ₓ_{₂}—O—SO₃-M;
R₂ is hydrogen; an unsubstituted or substituted alkyl or aryl; a radical of formula

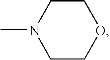

—OH; —NH₂; —N(CH₂CH₂OH)₂; —N[CH₂CH(OH)CH₃]₂; —NH—R₃; —N(R₃)₂ or —OR₃; or
R₁ and R₂ are each independently of the other are —OH; —Cl; —NH₂; —O—C₁-C₄alkyl; —O-aryl;

—NH—C₁-C₄alkyl; —N(C₁-C₄alkyl)₂; —N(C₁-C₄alkyl)(hydroxy-C₁-C₄alkyl); —N(hydroxy-C₁-C₄alkyl)₂; —NH-aryl; morpholino; or S—C₁-C₄alkyl(aryl);
R₈ and R₉ are each independently of the other hydrogen; C₁-C₄alkyl; phenyl; or a radical of formula;

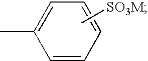

R₁₀ is hydrogen; Cl; or SO₃M;
R₁₁ is —CN; —SO₃M; —N(C₁-C₄alkyl)₂; or N(aryl)₂;
R₁₂ is hydrogen; —SO₃M; —O—C₁-C₄alkyl; —CN; —Cl; —COO—C₁-C₄alkyl; or CON(C₁-C₄alkyl)₂;
R₁₃ is hydrogen; —C₁-C₄alkyl; —Cl; or —SO₃M;
R₁₄ and R₁₅ are each independently of the other hydrogen; C₁-C₄alkyl; —SO₃M; —Cl; or —O—C₁-C₄alkyl;
R₁₆ is hydrogen; or C₁-C₄alkyl;
R₁₇ is hydrogen; C₁-C₄alkyl; —CN; —Cl; —COO—C₁-C₄alkyl; —CON(C₁-C₄alkyl)₂; aryl; or —O-aryl;
M is hydrogen; Na; K; Ca; Mg; ammonium; mono-, di-, tri- or tetra-C₁-C₄alkylammonium; mono-, di- or tri-C₁-C₄hydroxyalkylammonium; or ammonium di- or tri-substituted by a mixture of C₁-C₄alkyl and C₁-C₄hydroxyalkyl groups;
n₁, n₂ and n₃ are each independently of the others 0; or 1;
x₁ is 1; or 2; and
x₂ is from 1 to 3.

R₂, R₃, R₄, R₅, R₆ and R₇ in the meaning of (unsubstituted or) substituted alkyl are C₁-C₁₂alkyl, preferably C₁-C₄alkyl. The alkyl groups can be branched or unbranched and unsubstituted or substituted by halogen, e.g. fluorine, chlorine or bromine, C₁-C₄alkoxy, e.g. methoxy or ethoxy, phenyl or carboxyl, C₁-C₄alkoxycarbonyl, e.g. acetyl, mono- or di-C₁-C₄alkylamino or by —SO₃M.

R₂, R₃, R₄, R₅, R₆ and R₇ in the meaning of (unsubstituted or) substituted aryl are preferably a phenyl or naphthyl group which may be unsubstituted or substituted by C₁-C₄alkyl, e.g. methyl, ethyl, propyl, isopropyl, butyl, isobutyl, sec-butyl or tert-butyl, C₁-C₄alkoxy, e.g. methoxy, ethoxy, propoxy, isopropoxy, butoxy, isobutoxy, sec-butoxy or tert-butoxy, halogen, e.g. fluorine, chlorine or bromine, C₂-C₅alkanoylamino, e.g. acetylamino, propionylamino or butyrylamino, nitro, sulfo or by di-C₁-C₄alkylated amino.

The compounds of formula (FW-01) are preferably used in neutral form, that is to say M is preferably a cation of an alkali metal, especially sodium, or an amine.

In the compounds of formula (FW-01), R₁ is preferably a radical of formula

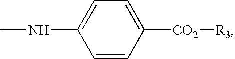

wherein R₃ is as defined above and is preferably C₁-C₄alkyl, especially methyl or ethyl; or a radical of formula

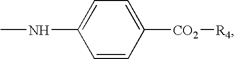

wherein R₄ is as defined above and is preferably C₁-C₄alkyl, especially methyl or ethyl, or —NR₆R₇, wherein R₆ and R₇ are as defined above and are preferably hydrogen, C₁-C₄alkyl, especially methyl or ethyl, a morpholino or piperidino radical, more especially hydrogen, or a radical of formula

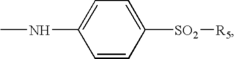

wherein R₅ is as defined above and is preferably C₁-C₄alkyl, especially methyl or ethyl substituted —SO₃M, wherein M is as defined above and is preferably sodium; and

R₂ is preferably

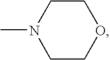

### —NH₂, —N(CH₂CH₂OH)₂ or —N[CH₂CH(OH)CH₃]₂.

The fluorescent whiteners that are advantageously suitable for use in the present invention are listed by way of example in Table 1 below:

TABLE 1
fluorescent whiteners

| Com- | |
|---|---|
| pound | |
| of | |
| formula | |
| (FW-09) | 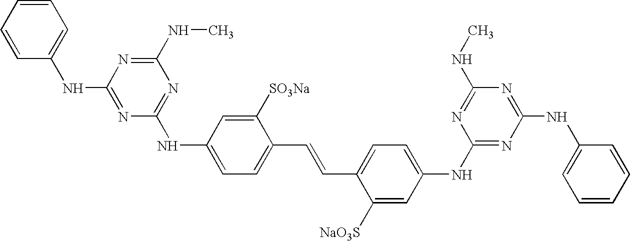 |
| (FW-10) | 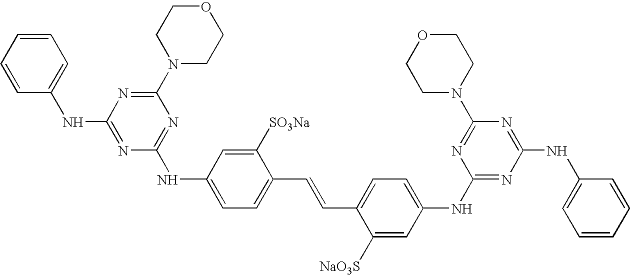 |
| (FW-11) | 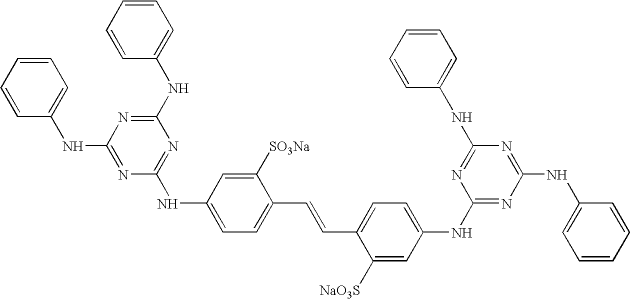 |
| (FW-12) | 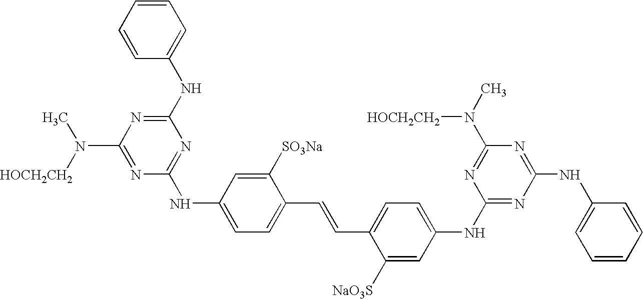 |
| (FW-13) | 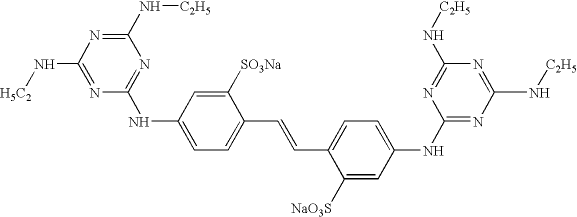 |
| (FW-14) | 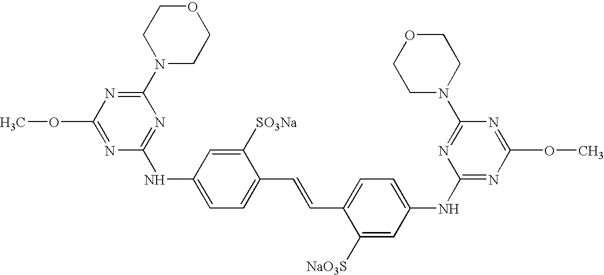 |
| (FW-15) | 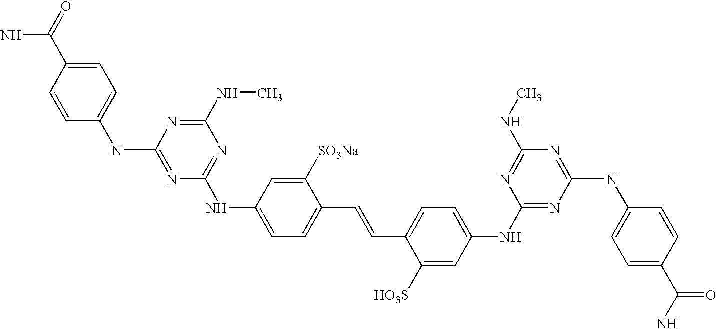 |
| (FW-16) | 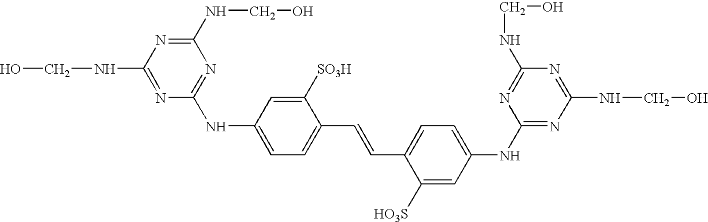 |
| (FW-17) | 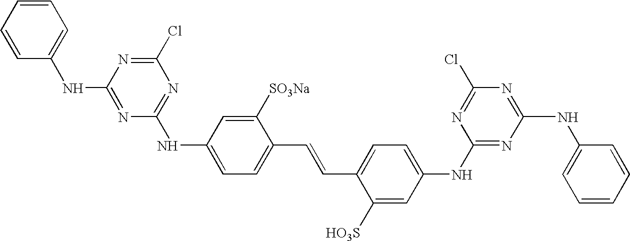 |
| (FW-18) | 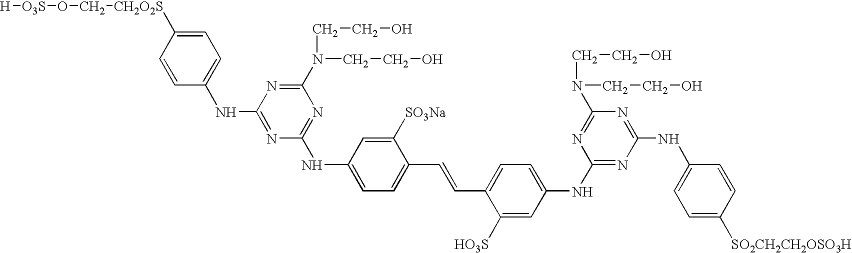 |
| (FW-19) | 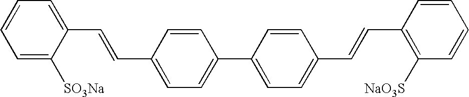 |
| (FW-20) | 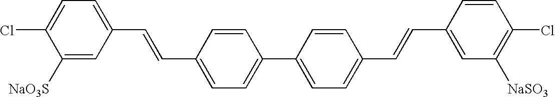 |
| (FW-21) | 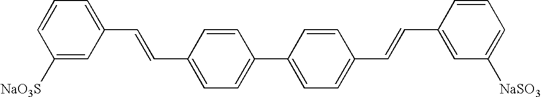 |
| (FW-22) | 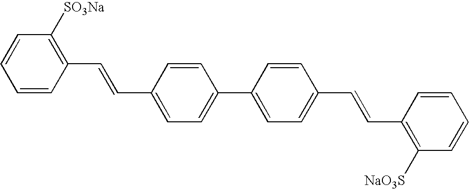 |
| (FW-23) | 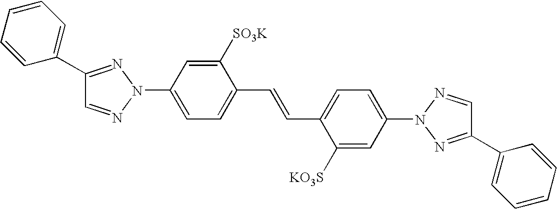 |
| (FW-24) | 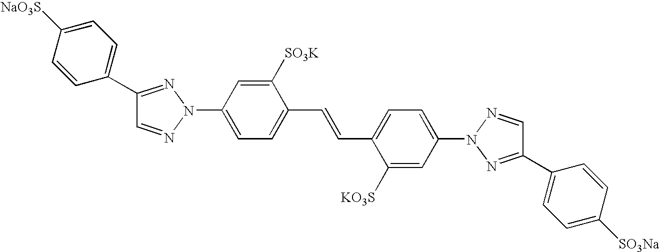 |
| (FW-25) | 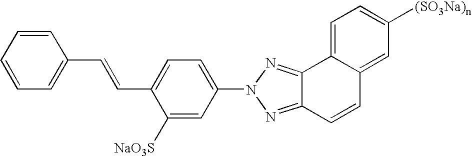 |
| (FW-26) | 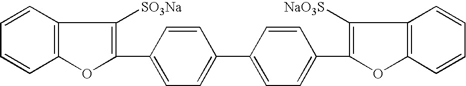 |
| (FW-27) | 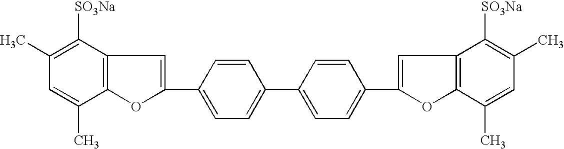 |
| (FW-28) | 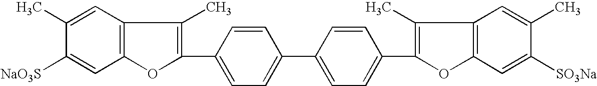 |
| (FW-29) | 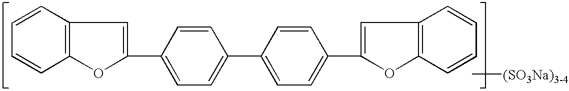 |
| (FW-30) | 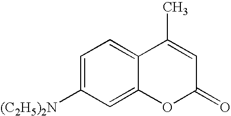 |

As component (b) there is preferably used kojic acid, α-arbutin, quercitin, aloesin, azelaic acid, guaiol, ellagic acid and esters thereof and also the fluorescent whiteners of formula (FW-22).

Each of the above-mentioned inhibitors can be used in the composition according to the invention singly or in admixture with one another.

It is also possible to use more than one of the additional skin-lightening compounds, such as two, three or four further compounds of component (b).

Preferably the ratio of components (a):(b) is 1:99, especially 5:95, and more especially from 10:90 to 99:1, preferably 95:5, and especially 90:10% by weight of component (b).

Special preference is given to mixtures of (a) and (b) wherein the ratio (a):(b) is from 20:80, preferably 30:70 to 80:20, especially 70:30.

The last-mentioned compositions can be used inter alia to improve solubility or to increase the skin-lightening action.

Furthermore, the compositions used according to the invention may comprise as component (c) one or more UV-A or UV-B absorbers of the following substance classes:

TABLE 2
Suitable UV filter substances which can be additionally used
with the UV absorbers according to the present invention
(The generic scope of the UV absorbers is described in the left-hand
column; specific compounds are indicated in the right-hand column)

p-aminobenzoic acid derivatives, for example 4-dimethylaminobenzoic acid 2-ethylhexyl
ester;
salicylic acid derivatives, for example salicylic acid 2-ethylhexyl ester;
benzophenone derivatives, for example 2-hydroxy-4-methoxybenzophenone and its
5-sulfonic acid derivative;
diphenylacrylates, for example 2-ethylhexyl 2-cyano-3,3-diphenylacrylate, and 3-(benzo-
furanyl) 2-cyanoacrylate;
3-imidazol-4-ylacrylic acid and esters;
benzofuran derivatives, especially 2-(p-aminophenyl)benzofuran derivatives, described in
EP-A-582 189, US-A-5 338 539, US-A-5 518 713 and EP-A-613 893;
polymeric UV absorbers, for example the benzylidene malonate derivatives described in
EP-A-709 080;
cinnamic acid derivatives, for example the 4-methoxycinnamic acid 2-ethylhexyl ester and
isoamyl ester or cinnamic acid derivatives described in US-A-5 601 811 and WO 97/00851;
camphor derivatives, for example 3-(4′-methyl)benzylidene-bornan-2-one, 3-benzylidene-
bornan-2-one, N-[2(and 4)-2-oxyborn-3-ylidene-methyl)-benzyl]acrylamide polymer, 3-(4′-
trimethylammonium)-benzylidene-bornan-2-one methyl sulfate, 3,3′-(1,4-phenylene-
dimethine)-bis(7,7-dimethyl-2-oxo-bicyclo[2.2.1]heptane-1-methanesulfonic acid) and salts,
3-(4′-sulfo)benzylidene-bornan-2-one and salts; camphorbenzalkonium methosulfate;
hydroxyphenyltriazine compounds, for example 2-(4′-methoxyphenyl)-4,6-bis(2′-hydroxy-4′-
n-octyloxyphenyl)-1,3,5-triazine; 2,4-bis{[4-(3-(2-propyloxy)-2-hydroxy-propyloxy)-2-
hydroxy]-phenyl}-6-(4-methoxyphenyl)-1,3,5-triazine; 2,4-bis{[4-(2-ethyl-hexyloxy)-2-
hydroxy]-phenyl}-6-[4-(2-methoxyethyl-carboxyl)-phenylamino]-1,3,5-triazine; 2,4-bis{[4-
(tris(trimethylsilyloxy-silylpropyloxy)-2-hydroxy]-phenyl}-6-(4-methoxyphenyl)-1,3,5-triazine;
2,4-bis{[4-(2″-methylpropenyloxy)-2-hydroxy]-phenyl}-6-(4-methoxyphenyl)-1,3,5-triazine;
2,4-bis{[4-(1′,1′,1′,3′,5′,5′,5′-heptamethyltrisilyl-2″-methyl-propyloxy)-2-hydroxy]-phenyl}-6-
(4-methoxyphenyl)-1,3,5-triazine; 2,4-bis{[4-(3-(2-propyloxy)-2-hydroxy-propyloxy)-2-
hydroxy]-phenyl}-6-[4-ethylcarboxy)-phenylamino]-1,3,5-triazine;
physical sunscreens, coated or not coated, such as titanium dioxide, zinc oxide, iron
oxides, mica, MnO, Fe₂O₃, Ce₂O₃, Al₂O₃, ZrO₂ (surface coatings: polymethylmethacrylate,
methicone (methylhydrogenpolysiloxane as described in CAS 9004-73-3), dimethicone,
isopropyl titanium triisostearate (as described in CAS 61417-49-0), metal soaps such as
magnesium stearate (as described in CAS 4086-70-8), perfluoroalcohol phosphate such as
C₉-C₁₅ fluoroalcohol phosphate (as described in CAS 74499-44-8; JP 5-86984; JP 4-
330007)). The primary particle size is, on average, 15 nm-35 nm and the particle size
distribution is in the range 100 nm-300 nm.
aminohydroxy-benzophenone derivatives disclosed in DE 100 11 317, EP 1 133 980 and
EP 1 046 391
phenyl-benzimidazole derivatives as disclosed in EP 1 167 358

The UV absorbers described in “Sunscreens”, Eds. N. J. Lowe, N. A. Shaath, Marcel Dekker, Inc., New York and Basel or in Cosmetics & Toiletries (107), 50ff (1992) can also be used as additional UV-protective substances.

Especially preferred are the light-protective substances indicated in Table 3 below:

TABLE 3
Suitable UV filter substances which can be additionally used
with the UV absorbers according to the present invention
(The generic scope of the UV absorbers is described in the left-hand
column; specific compounds are indicated in the right-hand column)

| No. | Chemical Name | CAS No. |
|---|---|---|
| 1 | (+/−)-1,7,7-trimethyl-3-[(4-methylphenyl)methylene]bicyclo- | 36861-47-9 |
| | [2.2.1]heptan-2-one | |
| 2 | 1,7,7-trimethyl-3-(phenylmethylene)bicyclo[2.2.1]heptan-2-one | 15087-24-8 |
| 3 | (2-hydroxy-4-methoxyphenyl)(4-methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-hydroxy-4-methoxybenzophenone | 131-57-7 |
| 7 | 2-hydroxy-4-methoxybenzophenone-5-sulfonic acid | 4065-45-6 |
| 8 | 2,2′-dihydroxy-4,4′-dimethoxybenzophenone | 131-54-4 |
| 9 | 2,2′-dihydroxy-4-methoxybenzophenone | 131-53-3 |
| 10 | alpha-(2-oxoborn-3-ylidene)toluene-4-sulfonic acid and its salts | 56039-58-8 |
| 11 | 1-[4-(1,1-dimethylethyl)phenyl]-3-(4-methoxyphenyl)propane-1,3- | 70356-09-1 |
| | dione | |
| 12 | methyl N,N,N-trimethyl-4-[(4,7,7-trimethyl-3-oxobicyclo[2.2.1]hept-2- | 52793-97-2 |
| | ylidene)methyl]anilinium sulphate; | |
| 22 | 3,3,5-trimethyl-cyclohexyl-2-hydroxy-benzoate | 118-56-9 |
| 23 | isopentyl p-methoxycinnamate | 71617-10-2 |
| 27 | menthyl o-aminobenzoate | 134-09-8 |
| 28 | menthyl salicylate | 89-46-3 |
| 29 | 2-ethylhexyl 2-cyano-3,3-diphenylacrylate | 6197-30-4 |
| 30 | 2-ethylhexyl 4-(dimethylamino)benzoate | 21245-02-3 |
| 31 | 2-ethylhexyl 4-methoxycinnamate | 5466-77-3 |
| 32 | 2-ethylhexyl salicylate | 118-60-5 |
| 33 | benzoic acid, 4,4′,4″-(1,3,5-triazine-2,4,6-triyltriimino)tris-, tris(2- | 88122-99-0 |
| | ethylhexyl) ester; 2,4,6-trianilino-(p-carbo-2′-ethylhexyl-1′-oxy)-1,3,5- | |
| | triazine | |
| 34 | 4-aminobenzoic acid | 150-13-0 |
| 35 | benzoic acid, 4-amino-, ethyl ester, polymer with oxirane | 113010-52-9 |
| 38 | 2-phenyl-1H-benzimidazole-5-sulphonic acid | 27503-81-7 |
| 39 | 2-propenamide, N-[[4-[(4,7,7-trimethyl-3-oxobicyclo[2.2.1]hept-2- | 147897-12-9 |
| | ylidene)methyl]phenyl]methyl]-, homopolymer | |
| 40 | triethanolamine salicylate | 2174-16-5 |
| 41 | 3,3′-(1,4-phenylenedimethylene)bis[7,7-dimethyl-2-oxo-bicyclo- | 90457-82-2 |
| | [2.2.1]heptane-1-methanesulfonic acid] | |
| 42 | titanium dioxide | 13463-67-7 |
| 44 | zinc oxide | 1314-13-2 |
| 45 | 2,2′-methylene-bis[6-(2H-benzotriazol-2-yl)-4-(1,1,3,3-tetramethyl- | 103597-45-1 |
| | butyl)-phenol] | |
| 46 | 2,4-bis{[4-(2-ethylhexyloxy)-2-hydroxy]-phenyl}-6-(4-methoxy- | 187393-00-6 |
| | phenyl)-(1,3,5)-triazine | |
| 47 | 1H-benzimidazole-4,6-disulfonic acid, 2,2′-(1,4-phenylene)bis-, | 180898-37-7 |
| | disodium salt | |
| 48 | benzoic acid, 4,4′-[[6-[[4-[[(1,1-dimethylethyl)amino]carbonyl]- | 154702-15-5 |
| | phenyl]amino]1,3,5-triazine-2,4-diyl]diimino]bis-, bis(2-ethylhexyl) | |
| | ester | |
| 49 | phenol, 2-(2H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]- | |
| 50 | dimethicodiethylbenzalmalonate | 207574-74-1 |
| 51 | benzenesulfonic acid, 3-(2H-benzotriazol-2-yl)-4-hydroxy-5-(1- | 92484-48-5 |
| | methylpropyl)-, monosodium salt | |
| 52 | benzoic acid, 2-[4-(diethylamino)-2-hydroxybenzoyl]-, hexyl ester | 302776-68-7 |
| 53 | 1-dodecanaminium, N-[3-[[4-(dimethylamino)benzoyl]amino]propyl]- | 156679-41-3 |
| | N,N-dimethyl-, salt with 4-methylbenzenesulfonic acid (1:1) | |
| 54 | 1-propanaminium, N,N,N-trimethyl-3-[(1-oxo-3-phenyl-2-propenyl)- | 177190-98-6 |
| | amino]-, chloride | |
| 55 | 1H-benzimidazole-4,6-disulfonic acid, 2,2′-(1,4-phenylene)bis- | 170864-82-1 |
| 56 | 1,3,5-triazine, 2,4,6-tris(4-methoxyphenyl)- | 7753-12-0 |
| 57 | 1,3,5-triazine, 2,4,6-tris[4-[(2-ethylhexyl)oxy]phenyl]- | 208114-14-1 |
| 58 | 1-propanaminium, 3-[[3-[3-(2H-benzotriazol-2-yl)-5-(1,1- | 340964-15-0 |
| | dimethylethyl)-4-hydroxyphenyl]-1-oxopropyl]amino]-N,N-diethyl-N- | |
| | methyl-, methyl sulfate (salt) | |
| 59 | 2-propenoic acid, 3-(1H-imidazol-4-yl)- | 104-98-3 |
| 60 | benzoic acid, 2-hydroxy-, [4-(1-methylethyl)phenyl]methyl ester | 94134-93-7 |
| 61 | 1,2,3-propanetriol, 1-(4-aminobenzoate) | 136-44-7 |
| 62 | benzeneacetic acid, 3,4-dimethoxy-□-oxo- | 4732-70-1 |
| 63 | 2-propenoic acid, 2-cyano-3,3-diphenyl-, ethyl ester | 5232-99-5 |
| 64 | anthranilic acid, p-menth-3-yl ester | 134-09-8 |
| 65 | 2,2′-bis(1,4-phenylene)-1H-benzimidazole-4,6-disulphonic acid | 349580-12-7 |
| | monosodium salt or disodium phenyl dibenzimidazole tetrasulfonate | |
| | or Neo-Heliopan AP | |

Each of the above-mentioned light-protective substances, especially the light-protective substances mentioned as being preferred in Table 3, can be used singly or in admixture with one another, for example two, three, four, five or six of the light-protective substances indicated in the Table.

Some of the UV absorbers listed in the Table above can simultaneously be used as solvents for cosmetic UV absorbers.

Preferred as component (c) in the composition used according to the invention are triazine UV absorbers of formula

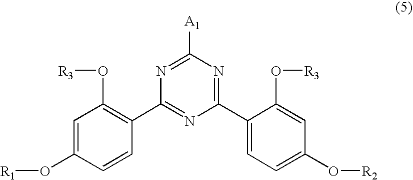

wherein
R₁ and R₂ are each independently of the other C₃-C₁₈alkyl; C₂-C₁₈alkenyl; a radical of formula —CH₂—CH(—OH)—CH₂—O-T₁; or
R₁ and R₂ are a radical of formula

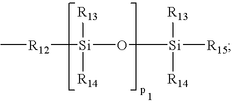

R₁₂ is a direct bond; a straight-chain or branched C₁-C₄alkylene radical or a radical of formula —Cₘ_{₁}H₂ₘ_{₁}— or —Cₘ_{₁}H₂ₘ_{₁}—O—;
R₁₃, R₁₄ and R₁₅ are each independently of the others C₁-C₁₈alkyl; C₁-C₁₈alkoxy or a radical of formula

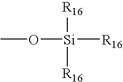

R₁₆ is C₁-C₅alkyl;
m₁ and m₃ are each independently of the other from 1 to 4;
p₁ is 0 or a number from 1 to 5;
A₁ is a radical of formula or of

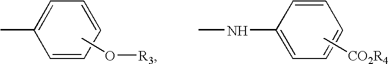

formula

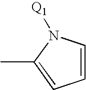

R₃ is hydrogen; C₁-C₁₀alkyl, —(CH₂CHR₅—O) n-R₄;or a radical of formula —CH₂—CH(—OH)—CH₂—O-T₁;
R₄ is hydrogen; M; C₁-C₅alkyl; or a radical of formula —(CH₂)ₘ_{₂}—O-T₁;
R₅ is hydrogen; or methyl;
T₁ is hydrogen; or C₁-C₈alkyl;
Q₁ is C₁-C₁₈alkyl;
M is a metal cation;
is from 1 to 4; and
n₁ is 1-16.

Special preference is given to the compounds of formulae

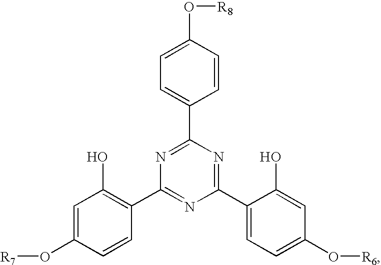

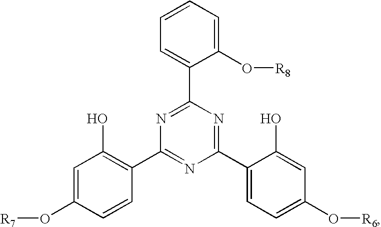

of formula

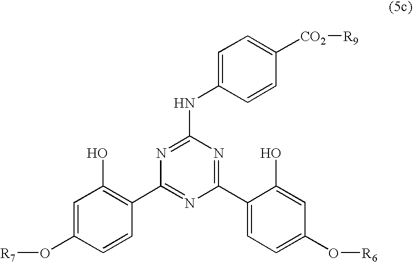

or of formula

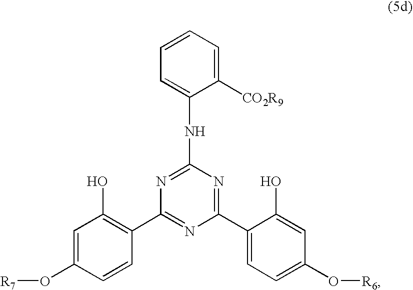

wherein
R₆ and R₇ are each independently of the other C₃-C₁₈alkyl; or —CH₂—CH(—OH)—CH₂—O-T₁;
R₈ is C₁-C₁₀alkyl or a radical of formula

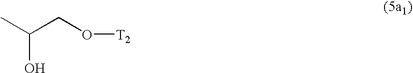

or of formula

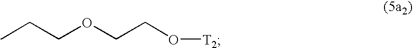

R₉ is hydrogen; M; C₁-C₅alkyl; or a radical of formula —(CH₂)ₘ—O-T₂
T₁ and T₂ are each independently of the other hydrogen; or C₁-C₅alkyl; and
m is from 1 to 4.

Of primary interest are compounds of formulae (5a) and (5b) wherein
R₆ and R₇ are each independently of the other C₃-C₁₈alkyl; or —CH₂—CH(—OH)—CH₂—O-T₁;
R₈ is C₁-C₁₀alkyl;
   and also compounds of formulae (5a) and (5b) wherein
R₆ and R₇ are each independently of the other C₃-C₁₈alkyl or —CH₂—CH(—OH)—CH₂—O-T₁; and
T₁ is hydrogen; or C₁-C₅alkyl.

Examples of compounds of formula (5) that may be mentioned are:
2-(4′-methoxyphenyl)-4,6-bis(2′-hydroxy-4′-n-octyloxyphenyl)-1,3,5-triazine;
2,4-bis{[4-(3-(2-propyloxy)-2-hydroxy-propyloxy)-2-hydroxy]-phenyl}-6-(4-methoxyphenyl)-1,3,5-triazine;
2,4-bis{[4-(2-ethyl-hexyloxy)-2-hydroxy]-phenyl}-6-[4-(2-methoxyethyl-carboxyl)-phenyl-amino]-1,3,5-triazine;
2,4-bis{[4-(tris(trimethylsiloxy-silylpropyloxy)-2-hydroxy]-phenyl}-6-(4-methoxyphenyl)-1,3,5-triazine;
2,4-bis{[4-(2″-methylpropenyloxy)-2-hydroxy]-phenyl}-6-(4-methoxyphenyl)-1,3,5-triazine;
2,4-bis{[4-(1′,1′,1′,3′,5′,5′,5′-heptamethyltrisilyl-2″-methyl-propyloxy)-2-hydroxy]-phenyl}-6-(4-methoxyphenyl)-1,3,5-triazine;
2,4-bis{[4-(3-(2-propyloxy)-2-hydroxy-propyloxy)-2-hydroxy]-phenyl}-6-[4-ethylcarboxyl)-phenylamino]-1,3,5-triazine; and
2,4-bis{[4-(2-ethyl-hexyloxy)-2-hydroxy]-phenyl}-6-(1-methylpyrrol-2-yl)-1,3,5-triazine.

Very special preference is given to those triazine compounds of formula (5) in which R₆ and R₇ have the same meanings and especially the compound of formula

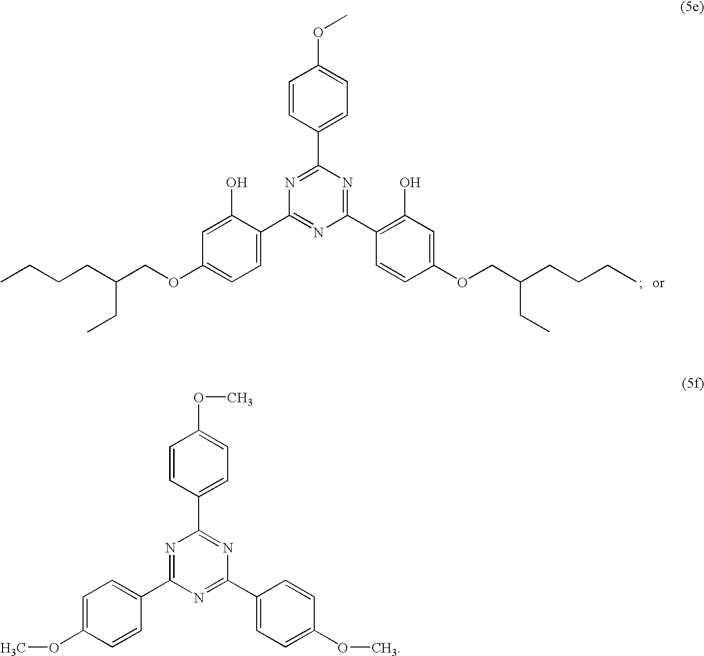

According to the invention there are also used as organic UV absorbers benzotriazole compounds of formula

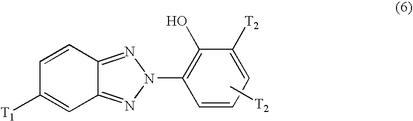

wherein
T₁ is hydrogen; or C₁-C₃alkyl and
T₂ is unsubstituted C₁-C₄alkyl, preferably tert-butyl, or phenyl-substituted C₁-C₄alkyl, preferably α,α-dimethylbenzyl.

Preferred benzotriazole compounds used according to the invention correspond to formula

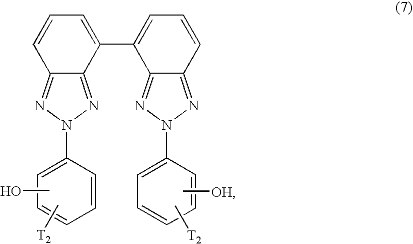

wherein
T₂ is hydrogen; or C₁-C₁₂alkyl, preferably iso-octyl.

Also preferred are benzotriazole compounds of formula

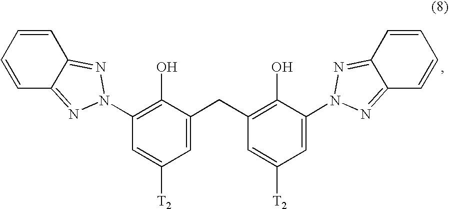

wherein
T₂ is C₁-C₁₂alkyl, preferably iso-octyl.

Further preferred compounds corresponding to component (c) used according to the invention are octyl methoxycinnamate and benzophenone-3.

In addition to components (a), (b) and (c), in the composition according to the invention it is also possible to use, as component (d), secondary light-protective substances of the antioxidant type which interrupt the photochemical reaction chain triggered when UV radiation penetrates the skin or hair. This property is desirable in cosmetic light-protection because the action of UV and light can bring about the formation of harmful free radicals both in formulations and on the skin. By providing the compositions according to the invention with antioxidants, in addition to the protection against UV damage there is simultaneously achieved protection against photochemical degradation of ingredients in the formulation. Typical examples of such antioxidants are:
amino acids (e.g. histidine, tyrosine, tryptophan, phenyl aniline) and derivatives thereof,
imidazoles (e.g. urocanic acid) and derivatives thereof,
peptides such as D,L-carnosine, D-carnosine, L-carnosine and derivatives thereof (e.g. anserine),
carotinoids, carotenes (e.g. α-carotene, β-carotene, lycopene) and derivatives thereof,
chlorogenic acid and derivatives thereof,
lipoic acid and derivatives thereof (e.g. dihydrolipoic acid),
aurothioglycose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, α-linoleyl, cholesteryl and glyceryl esters thereof) and salts thereof,
dilaurylthiodipropionate, distearylthiodipropionate, thiodipropionic acid and derivatives thereof (esters, ethers, peptides, lipids, nucleotides, nucleosides and salts) and also
sulfoximine compounds (e.g. buthionine sulfoximines, homocysteine sulfoximine, butionine sulfones, penta-, hexa-, hepta-thionine sulfoximine) in very small tolerable amounts, and also
(metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. α-linolenic acid, linoleic acid, oleic acid), folic acid and derivatives thereof, ubiquinone and ubiquinol and derivatives thereof, tocopherols and derivatives (e.g. vitamin E acetate), vitamin A and derivatives (e.g. vitamin A palmitate) and also coniferyl benzoate of benzoic resin, rutic acid and derivatives thereof, α-glycosylrutin, ferulic acid, furfurylidene glucitol, carnosine, butylhydroxytoluene, butylhydroxyanisole, nordihydroguaiac resin acid, nordihydroguaiaretic acid, trihydroxy-butyrophenone, uric acid and derivatives thereof, mannose and derivatives thereof, superoxide dismutase, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients.

Mention may also be made of the phenolic antioxidants listed in the following Table 4:

TABLE 4
Antioxidants

| Compound of | |
|---|---|
| formula | |
| (AO 1) | 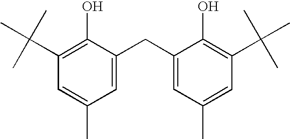 |
| (AO 2) | 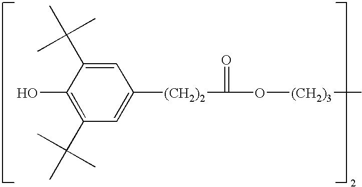 |
| (AO 3) | 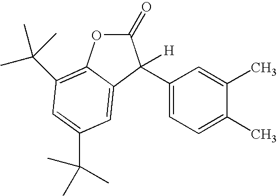 |
| (AO 4) | 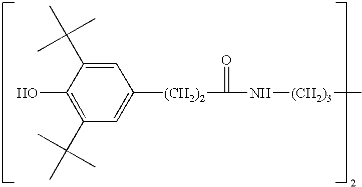 |
| (AO 5) | 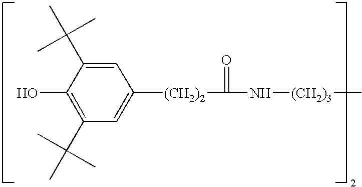 |
| (AO 6) | 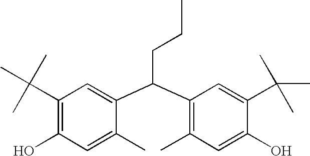 |
| (AO 7) | 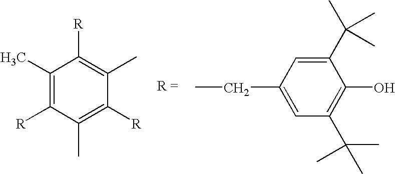 |
| (AO 8) | 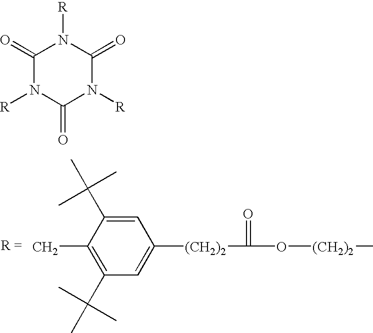 |
| (AO 9) | 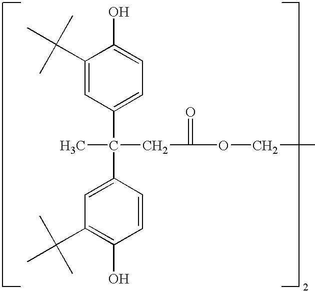 |
| (AO 10) | 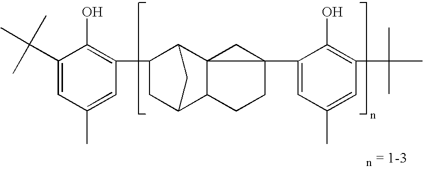 |
| (AO 11) | 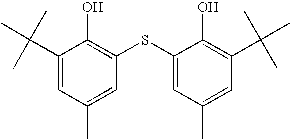 |
| (AO 12) | 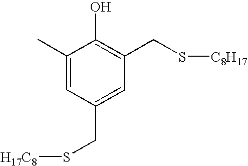 |
| (AO 13) | 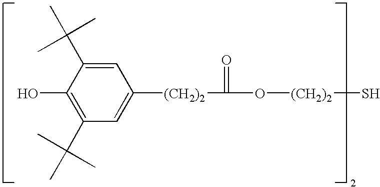 |
| (AO 14) | 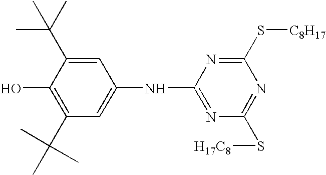 |
| (AO 15) | 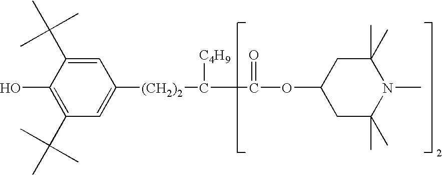 |
| (AO 16) | 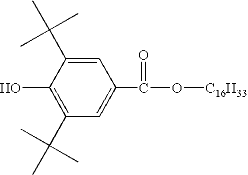 |
| (AO 17) | 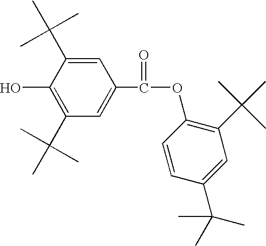 |
| (AO 18) | 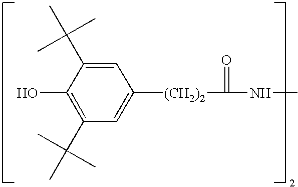 |
| (AO 19) | 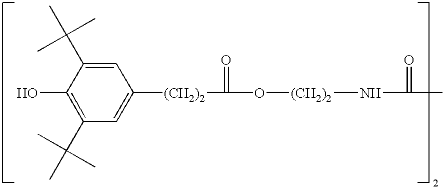 |
| (AO 20) | 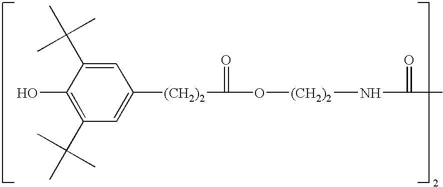 |
| (AO 21) | 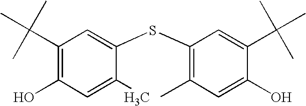 |
| (AO 22) | 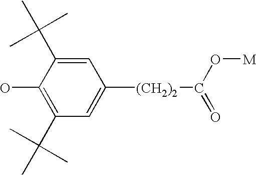 |
| (AO 23) | 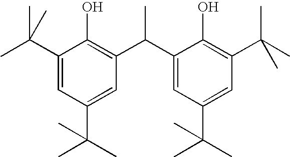 |
| (AO 24) | 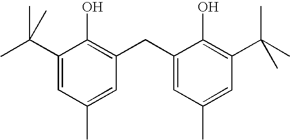 |
| (AO 25) | 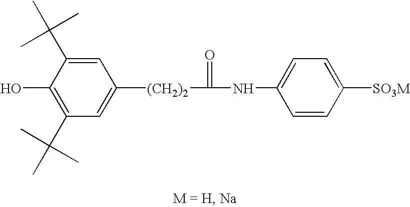 |
| (AO 26) | 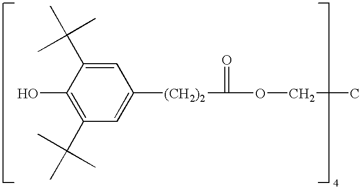 |
| (AO 27) | 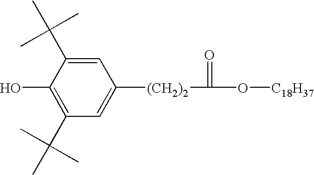 |
| (AO 28) | 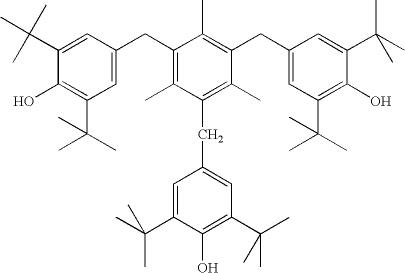 |

For the composition according to the invention there also come into consideration sterically hindered amines (also referred to as HALS compounds (=“Hindered Amine Light Stabilisers”)).

Such a compound is preferably a 2,2,6,6-tetraalkylpiperidine derivative.

Examples of tetraalkylpiperidine derivatives suitable for use according to the invention can be found in EP-A-356 677, pages 3-17, sections a) to f). The said sections of that EP-A are to be regarded as part of this description. It is especially advantageous to use the following tetraalkylpiperidine derivatives:

bis(2,2,6,6-tetramethyl-piperidin-4-yl)sebacate, bis(2,2,6,6-tetramethyl-piperidin-4-yl)succinate, bis(1,2,2,6,6-pentamethylpiperidin-4-yl)sebacate, bis(1-octyloxy-2,2,6,6-tetramethylpiperidin-4-yl)sebacate, n-butyl-3,5-di-tert-butyl-4-hydroxybenzyl-malonic acid bis(1,2,2,6,6-pentamethylpiperidyl)ester, condensation product of 1-hydroxyethyl-2,2,6,6-tetramethyl-4-hydroxypiperidine and succinic acid, condensation product of N,N′-bis(2,2,6,6-tetramethyl-4-piperidyl)hexamethylenediamine and 4-tert-octylamino-2,6-dichloro-1,3,5-s-triazine, tris(2,2,6,6-tetramethyl-4-piperidyl)nitrilotriacetate, tetrakis(2,2,6,6-tetramethyl-4-piperidyl)-1,2,3,4-butanetetraoate, 1,1′-(1,2-ethanediyl)-bis(3,3,5,5-tetramethyl-piperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl)-malonate, 3-n-octyl-7,7,9,9-tetramethyl-1,3,8-triazaspiro[4.5]decane-2,4-dione, bis(1-octyloxy-2,2,6,6-tetramethylpiperidyl)sebacate, bis(1-octyloxy-2,2,6,6-tetramethylpiperidyl)succinate, condensation product of N,N-bis(2,2,6,6-tetramethyl-4-piperidyl)hexamethylenediamine and 4-morpholino-2,6-dichloro-1,3,5-triazine, condensation product of 2-chloro-4,6-di-(4-n-butylamino-2,2,6,6-tetramethylpiperidyl)-1,3,5-triazine and 1,2-bis(3-aminopropyl-amino)ethane, condensation product of 2-chloro-4,6-di-(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)-pyrrolidine-2,5-dione, 3-dodecyl-1-(1,2,2,6,6-pentamethyl-4-piperidyl)-pyrrolidine-2,5-dione, mixture of 4-hexadecyloxy- and 4-stearyloxy-2,2,6,6-tetramethylpiperidine, 2,6-dichloro-1,3,5-triazine, condensation product of 1,2-bis(3-aminopropylamino)ethane and 2,4,6-trichloro-1,3,5-triazine and also 4-butylamino-2,2,6,6-tetramethyl-piperidine (CAS Reg. No. [136504-96-6]); (2,2,6,6-tetramethyl-4-piperidyl)-n-dodecylsuccinimide, (1,2,2,6,6-pentamethyl-4-piperidyl)-n-dodecylsuccinimide, 2-undecyl-7,7,9,9-tetramethyl-1-oxa-3,8-diaza-4-oxo-spiro[4,5]decane, reaction product of 7,7,9,9-tetramethyl-2-cycloundecyl-1-oxa-3,8-diaza-4-oxospiro[4,5]decane and epichlorohydrin, tetra(2,2,6,6-tetramethylpiperidin-4-yl)butane-1,2,3,4-tetracarboxylate, tetra(1,2,2,6,6-pentamethylpiperidin-4-yl)butane-1,2,3,4-tetracarboxylate, 2,2,4,4-tetramethyl-7-oxa-3,20-diaza-21-oxo-dispiro[5.1.11.2]heneicosane, 8-acetyl-3-dodecyl-1,3,8-triaza-7,7,9,9-tetra-methylspiro[4,5]decane-2,4-dione, or a compound of formula

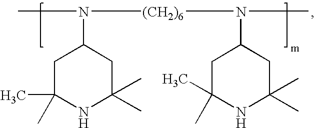

wherein m is a value of 5-50,

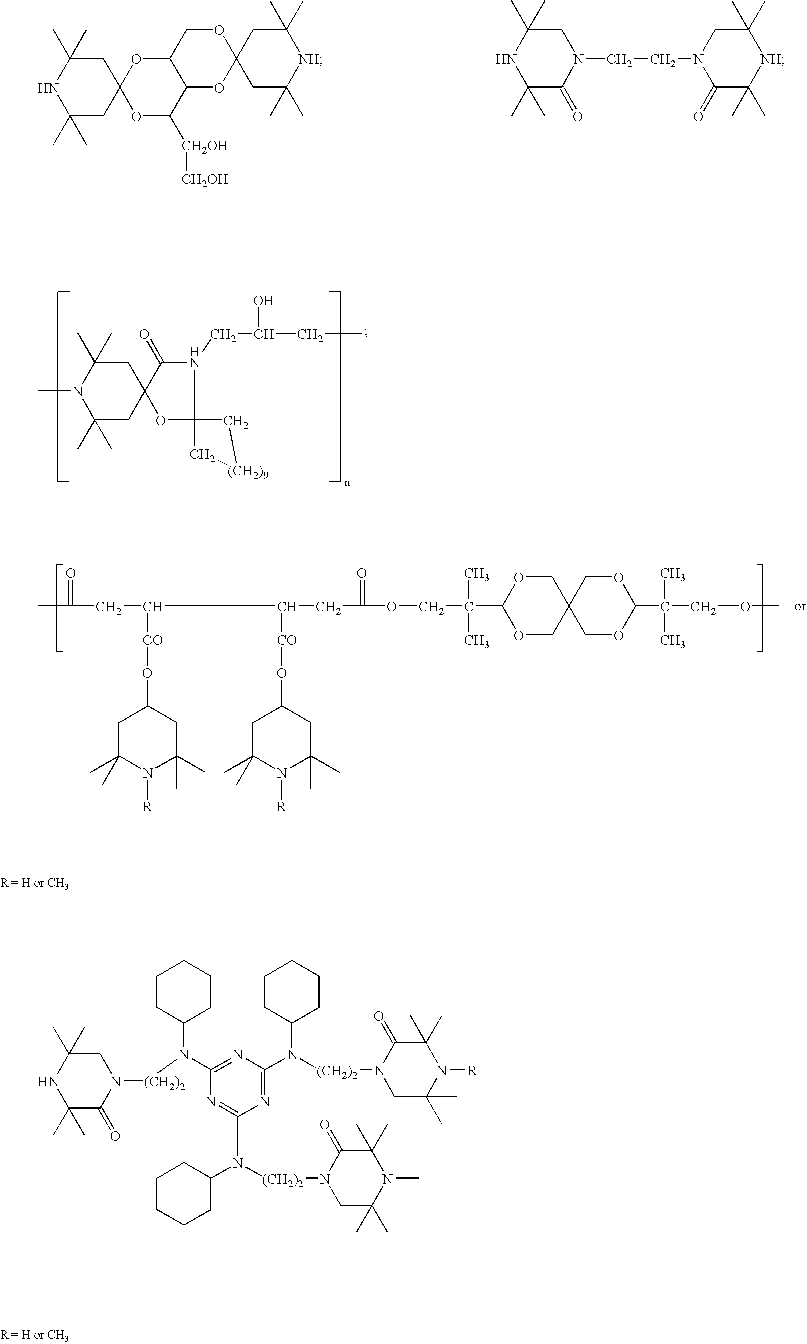

The proportion of antioxidants is usually from 0.001 to 30% by weight, preferably from 0.01 to 3% by weight, based on the weight of the composition according to the invention.

The additional substances of component (b), (c) or (d) may have different functions:

a. Direct influence on melanin synthesis, for example pyrone derivatives, especially kojic acid, 4-hydroxyphenyl-D-glucopyranoside (α- and β-arbutin), resorcinol derivatives, for example resorcinol, benzo[b]pyran derivatives, e.g. 8-α-glycopyranosyl-7-hydroxy-5-methyl-2-(2-oxopropyl)-4H-1-benzopyran-4-one (aloesin) or ellagic acid, azulene, guaiol and vitamin C;
b. Indirect influence on melanin synthesis via interacting cells, for example vitamin A, cytokines, prostaglandins or growth factors;
c. Indirect influence through mechanical removal of the dead skin, for example salicylic acid or lactates, e.g. lactic acid;
d. Indirect influence by UV protection: UV-A filters.
e. direct influence on skin lightening with fluorescent whiteners: by shifting of the L* value.

Table 5 below gives examples of mixtures of hydroxydiphenyl ether compounds of formula (1) (component (a)) with further skin-lightening active ingredients (component (b)) and UV absorbers (component (c)). The data relate to % by weight in the final cosmetic formulation.

TABLE 5

| Compo- | Formulation | | | | | | | | | | |
|---|---|---|---|---|---|---|---|---|---|---|---|
| nent | C1 | C2 | C3 | C4 | C5 | C6 | C7 | C8 | C9 | C10 | C11 |
| Compo- | 0.05 | 0.1 | 0.1 | 0.1 | 0.2 | 0.3 | 0.3 | 0.3 | 0.5 | 1 | |
| nent (a) | | | | | | | | | | | |
| (total) | | | | | | | | | | | |
| (a₁) | 0.05 | 0.1 | 0.1 | 0.12 | 0.2 | 0.3 | 0.3 | 0.3 | 0.5 | 1 | 0 |
| Compo- | 0.1 | 0.2 | 0.4 | 0.2 | 0.1 | 0.2 | 0.3 | 0.5 | 0.3 | 5 | 5 |
| nent (b) | | | | | | | | | | | |
| (total) | | | | | | | | | | | |
| (b₁) | 0.05 | 0.1 | 0.2 | | 0.1 | | | 0.2 | 0.3 | | |
| (b₂) | 0.05 | | 0.1 | 0.2 | | 0.1 | | 0.3 | | 2 | 2 |
| (b₃) | | 0.1 | | | | | 0.1 | | | | |
| (b₄) | | | 0.1 | | | 0.1 | | | | | |
| (b₅) | | 0.5 | | | | | | 0.3 | | | |
| (b₆) | | | | | | | | 0.2 | | | |
| (b₇) | | | 0.5 | | | | | | | | |
| (b₈) | | 2 | | | | | | | | | |
| (b₉) | 0.05 | | 0.1 | | | | | | | | |
| (b₁₀) | | | | | 0.1 | | | | | | |
| (b₁₁) | | | | | | | 0.2 | | | 3 | 3 |
| Compo- | 30 | 25 | 15 | 25 | 20 | 0 | 35 | 15 | 20 | 10 | 10 |
| nent (c) | | | | | | | | | | | |
| (total) | | | | | | | | | | | |
| (c₁) | 5 | | | | 5 | | | | | | |
| (c₂) | 2 | | | | | | 5 | | | 2 | 2 |
| (c₃) | | 10 | 5 | | | | | | | | |
| (c₄) | 10 | | | | 2 | | | | | | |
| (c₅) | 5 | | | | | | 5 | | | | |
| (c₆) | | | | | | | | | 5 | | |
| (c₇) | | | 5 | 10 | | | | 5 | | | |
| (c₈) | | | 5 | 5 | | | | | | | |
| (c₉) | | | | 5 | | | 5 | 5 | | | |
| (c₁₁) | | | | | 8 | | | | | | |
| (c₁₂) | | | | | | | 5 | | | | |
| (c₁₃) | | 5 | | | | | | | | | |
| (c₁₄) | | 5 | | 5 | | | 8 | | | | |
| (c₁₅) | | | | | 5 | | | | | | |
| (c₁₆) | | | | | | | 2 | | 2 | | |
| (c₁₇) | 3 | | | | | | | 5 | | | |
| (c₁₈) | 2 | | | | | | 5 | | | | |
| (c₁₉) | 2 | | | | | | | | 3 | | |
| (c₂₀) | | 3 | | | | | | | | 3 | 3 |
| (C₂₀) | | 2 | | | | | | | 5 | | |
| (c₂₁) | | | | | | | | | 2 | | |
| (c₂₂) | | | | | | | | | 3 | | |
| (c₂₃) | | | | | | | | | | 5 | 5 |
| Compo- | 1.1 | | 0.3 | | 0.1 | 0.2 | | | | 0.3 | 0.3 |
| nent (d) | | | | | | | | | | | |
| (total) | | | | | | | | | | | |
| (d₁) | 0.1 | | | | 0.1 | | | | | 0.1 | 0.1 |
| (d₂) | 1 | | | | | | | | | | |
| (d₃) | | | 0.3 | | | 0.2 | | | | 0.2 | 0.2 |

(a₁) Hydroxydiphenyl ether compound of formula (3)
(b₁) 5-HYDROXY-2-HYDROXY-METHYL-4H-PYRAN-4-ONE (=KOJIC ACID)[501-30-4]
(b₂) 4-HYDROXYPHENYL β-D-GLUCOPYRANOSIDE (=ARBUTIN) [497-76-7]
(b₃) (2,5-DIOXO-4-IMIDAZOLIDINYL)-UREA (=ALLANTOIN) [97-59-6]
(b₄) 2-(3,4-DIHYDROXYPHENYL)-3,5,7-TRIHYDROXY-4H-1-BENZOPYRAN-4-ONE (=QUERCITIN) [117-39-5]
(b₅) GUAIOL
(b₆) AZULENE
(b₇) SALICYLIC ACID
(b₈) LACTIC ACID
(b₉) RESORCINOL
(b₁₀) ALOESIN
(b₁₁) 1,7-HEPTANEDICARBOXYLIC ACID (=AZELAIC ACID) [123-99-9]
(C₁) BENZENESULFONIC ACID, 3-(2H-BENZOTRIAZOL-2-YL)-4-HYDROXY-5-(1-METHYL-PROPYL)-, MONOSODIUM SALT [92484-48-5]
(c₂) PROPYL GALLATE [121-79-9]
(C₃) N-[3-(3,5-DI-TERT-BUTYL-4-HYDROXYPHENYL)PROPIONYL] SULFANILICACID (OR SALTS, E.G. WITH SODIUM)
(c₄) BENZYLIDENE MALONATE POLYSILOXANE [207574-74-1]
(c₅) DROMETRIZOLE TRISILOXANE [155633-54-8] (15)
(c₆) DIETHYLHEXYL BUTAMIDO TRIAZONE [154702-15-5] (10)
(c₇) PHENOL, 2,2′-[6-(4-METHOXY-PHENYL)-1,3,5-TRIAZINE-2,4-DIYL]BIS[5-[(2-ETHYLHEXYL)OXY]- [187393-00-6] (10)
(c₈) 1H-BENZIMIDAZOLE-4,6-DISULFONIC ACID, 2,2′-(1,4-PHENYLENE)BIS-,DISODIUM SALT [180898-37-7] (10)
(c₉) BIS-BENZOTRIAZOLYL TETRA-METHYLBUTYL-PHENOL [103597-45-1] (10)
(c₁₀) TEREPHTHALYLIDENE (c10)DICAMPHOR SULFONIC ACID [90457-82-2](10)
(c₁₁) POLYACRYLAMIDOMETHYL BENZYLIDENE CAMPHOR [113783-61-2] (6)
(c₁₂) PHENYLBENZIMIDAZOLE SULFONIC ACID [27503-81-7] (8)
(c₁₃) ETHYLHEXYL METHOXYCINNA-MATE [5466-77-3] (10)
(c₁₄) OCTOCRYLENE [6197-30-4] (10)
(c₁₅) CAMPHOR BENZALKONIUM METHOSULFATE [52793-97-2] (6)
(c₁₆) BUTYL METHOXYDIBENZOYL-METHANE [70356-09-1] (5)
(c₁₇) BENZOPHENONE-3 [131-57-7](10)
(c₁₈) BENZOPHENONE-4 [4065-45-6](5)
(c₁₉) 1-DODECANAMINIUM, N-[3-[[4-(DIMETHYLAMINO)BENZOYL]-AMINO]PROPYL]-N,N-DIMETHYL-, SALT WITH 4-METHYL-BENZENE-SULFONIC ACID [156679-41-3]
(C₂₀) 1-PROPANAMINIUM, N,N,N-TRIMETHYL-3-[(1-OXO-3-PHENYL-2-PROPENYL)AMINO]-, CHLORIDE [177190-98-6]
(c₂₁) 3-BENZYLIDENE CAMPHOR [15087-24-8] (2)
(c₂₂) 4-METHYLBENZYLIDENE CAMPHOR [36861-47-9] (4)
(c₂₃) BENZYLIDENE CAMPHORSULFONIC ACID [56039-58-8] (6)
(d₁) Vitamin A
(d₂) Vitamin C
(d₃) Vitamin C palmitate

The halogenated hydroxydiphenyl ether compounds used according to the invention and mixtures of those compounds with one or more representatives of component (b) and/or (c) can be used as cosmetic formulations.

The invention therefore relates also to a cosmetic formulation which comprises

(a) a compound of formula (1); and optionally
(b) further skin-lightening active ingredients, and optionally
(c) one or more UV-A and/or UV-B absorbers, and optionally
(d) an antioxidant, and also
cosmetically acceptable adjuvants or carriers.

The cosmetic formulation according to the invention preferably comprises
from 0.001 to 10% by weight, preferably from 0.05 to 1% by weight, component (a),
from 0 to 10% by weight, preferably from 0.01 to 2% by weight, component (b),
from 0 to 30% by weight, preferably from 0.1 to 15% by weight, component (c), and
from 0 to 30% by weight, preferably from 0.01 to 5% by weight, component (d).

The invention relates also to a cosmetic formulation comprising
from 0.05 to 2% component (a), selected from the compound of formula (3) or (4),
from 0.001 to 2% component (b), selected from the compound of formula (FW 19),
from 0.01 to 2% component (d), selected from the compound of formula (AO 26) and (AO 27).

Such a formulation is preferably used in surfactant-containing cleansing compositions.

Another aspect of the present invention is therefore a cleaning composition, comprising

0.05 to 2% b.w. of component (a),
0.001 to 2% b.w. of component (b),
0 to 2% b.w. of component (c),
0 to 2% b.w. of component (d), and
0.1 to 10% b.w. of one or more synthetic detergents or soaps or a combination of such substances.

If desired, one or more compounds of components (a) to (d) may be present in special carriers, e.g. nanotopes, liposomes, glass particles, nanocolloids or emulsions.

The cosmetic formulations according to the invention are suitable for the protection of ultraviolet-sensitive organic materials, especially the skin, and for lightening the skin.

The compositions according to the invention can be used both in dissolved form and in the micronised state.

The cosmetic compositions can be prepared by physically mixing components (a), (b) and optionally (c) and (d) with the adjuvant using customary methods, e.g. by simply stirring the individual components together. The UV absorber can be used e.g. without further treatment or in the micronised state or in the form of a powder.

The cosmetic compositions can be, for example, creams, gels, lotions, alcoholic and aqueous/alcoholic solutions, sprays, mixed liposomes, liposome mixtures, emulsions, wax/fatty compositions, stick preparations, powders or ointments.

As water- and oil-containing emulsions (e.g. W/O, O/W, O/W/O and W/O/W emulsions or microemulsions) the compositions contain, for example,
from 0.1 to 30% by weight, preferably from 0.1 to 15% by weight and especially from 0.5 to 10% by weight, based on the total weight of the composition, of component (a) and optionally (b), (c) and (d),
from 1 to 60% by weight, especially from 5 to 50% by weight and preferably from 10 to 35% by weight, based on the total weight of the composition, of at least one oil component,
from 0 to 30% by weight, especially from 1 to 30% by weight und preferably from 4 to 20% by weight, based on the total weight of the composition, of at least one emulsifier,
from 10 to 90% by weight, especially from 30 to 90% by weight, based on the total weight of the composition, of water, and
from 0 to 88.9% by weight, especially from 1 to 50% by weight, of further cosmetically acceptable adjuvants.

As oil components of oil-containing compositions (e.g. oils, W/O, O/W, O/W/O and W/O/W emulsions or microemulsions) there come into consideration, for example, Guerbet alcohols based on fatty alcohols having from 6 to 18, preferably from 8 to 10, carbon atoms, esters of linear C₆-C₂₄ fatty acids with linear C₃-C₂₄ alcohols, esters of branched C₆-C₁₃-carboxylic acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, triglycerides based on C₆-C₁₀ fatty acids, liquid mono-/di-/tri-glyceride mixtures based on C₆-C₁₈ fatty acids, esters of C₆-C₂₄ fatty alcohols and/or Guerbet alcohols with aromatic carboxylic acids, especially benzoic acid, esters of C₂-C₁₂dicarboxylic acids with linear or branched alcohols having from 1 to 22 carbon atoms or polyols having from 2 to 10 carbon atoms and from 2 to 6 hydroxy groups, vegetable oils (such as sunflower oil, olive oil, soybean oil, rapeseed oil, almond oil, jojoba oil, orange oil, wheatgerm oil, peach kernel oil and the liquid components of coconut oil), branched primary alcohols, substituted cyclohexanes, linear and branched C₆-C₂₂ fatty alcohol carbonates, Guerbet carbonates, esters of benzoic acid with linear and/or branched C₆-C₂₂ alcohols (e.g. Finsolv® TN), linear or branched, symmetric or asymmetric dialkyl ethers having a total of from 12 to 36 carbon atoms, especially from 12 to 24 carbon atoms, for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, n-hexyl-n-undecyl ether, di-tert-butyl ether, diisopentyl ether, di-3-ethyldecyl ether, tert-butyl-n-octyl ether, isopentyl-n-octyl ether and 2-methyl-pentyl-n-octyl ether; ring-opening products of epoxidised fatty acid esters with polyols, silicone oils and/or aliphatic or naphthenic hydrocarbons. Also of importance are monoesters of fatty acids with alcohols having from 3 to 24 carbon atoms. That group of substances comprises the esterification products of fatty acids having from 8 to 24 carbon atoms, for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical mixtures thereof (obtained, for example, in the pressure removal of natural fats and oils, in the reduction of aldehydes from Roelen's oxosynthesis or in the dimerisation of unsaturated fatty acids) with alcohols, for example isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol and brassidyl alcohol and technical mixtures thereof (obtained, for example, in the high-pressure hydrogenation of technical methyl esters based on fats and oils or aldehydes from Roelen's oxosynthesis and as monomer fractions in the dimerisation of unsaturated fatty alcohols). Of special importance are isopropyl myristate, isononanoic acid C₁₆-C₁₈alkyl esters, stearic acid 2-ethylhexyl ester, cetyl oleate, glycerol tricaprylate, coconut fatty alcohol caprinate/caprylate and n-butyl stearate. Further oil components that can be used are dicarboxylic acid esters, such as di-n-butyl adipate, di(2-ethylhexyl)adipate, di(2-ethylhexyl)succinate and diisotridecyl acetate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate.

Preferred mono- or poly-ols are ethanol, isopropanol, propylene glycol, hexylene glycol, glycerol and sorbitol. It is also possible to use di- and/or tri-valent metal salts (alkaline earth metal, Al³⁺ inter alia) of one or more alkylcarboxylic acids.

The oil components can be used in an amount of, for example, from 1 to 60% by weight, especially from 5 to 50% by weight and preferably from 10 to 35% by weight, based on the total weight of the composition.

Any conventionally usable emulsifier can be used for the compositions.

As emulsifiers there come into consideration, for example, non-ionic surfactants from the following groups:
addition products of from 2 to 30 mol of ethylene oxide and/or from 0 to 5 mol of propylene oxide with linear fatty alcohols having from 8 to 22 carbon atoms, with fatty acids having from 12 to 22 carbon atoms and alkylphenols having from 8 to 15 carbon atoms in the alkyl group, for example ceteareth-20 or ceteareth-12;
C₁₂-C₂₂ fatty acid mono- and di-esters of addition products of from 1 to 30 mol of ethylene oxide with polyols having from 3 to 6 carbon atoms, especially with glycerol;
glycerol mono- and di-esters and sorbitan mono- and di-esters of saturated and unsaturated fatty acids having from 6 to 22 carbon atoms and ethylene oxide addition products thereof, for example glyceryl stearate, glyceryl isostearate, glyceryl oleate, sorbitan oleate or sorbitan sesquioleate;
C₈-C₂₂alkyl-mono- and -oligo-glycosides and ethoxylated analogues thereof, degrees of oligomerisation of from 1.1 to 5, especially from 1.2 to 1.4, being preferred, and glucose being preferred as the sugar component;
addition products of from 2 to 60 mol, especially from 15 to 60 mol, of ethylene oxide with castor oil and/or hardened castor oil;
polyol esters and especially polyglycerol esters, for example diisostearoyl polyglyceryl-3 diisostearate, polyglyceryl-3 diisostearate, triglyceryl diisostearate, polyglyceryl-2 sesquiisostearate or polyglyceryl dimerate. Mixtures of compounds from a plurality of those substance classes are also suitable;
partial esters based on linear, branched, unsaturated or saturated C₆-C₂₂ fatty acids, ricinoleic acid and also 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and also polyglucosides (e.g. cellulose), for example polyglyceryl-2-dihydroxystearates or polyglyceryl-2-diricinoleates;
mono-, di- and tri-alkylphosphates and also mono-, di- and/or tri-PEG-alkylphosphates and salts thereof;
wool wax alcohols;
one or more ethoxylated esters of natural derivatives, for example polyethoxylated esters of hydrogenated castor oil;
silicone oil emulsifiers, for example silicone polyol;
polysiloxane/polyalkyl/polyether copolymers and corresponding derivatives, for example cetyl dimethicone copolyol;
mixed esters of pentaerythritol, fatty acids, citric acid and fatty alcohol (see DE-A-1 165 574) and/or mixed esters of fatty acids having from 6 to 22 carbon atoms, methylglucose and polyols, preferably glycerol or polyglycerol, for example polyglyceryl-3 glucose distearate, polyglyceryl-3 glucose dioleate, methyl glucose dioleate or dicocoyl pentaerythryl distearyl citrate and also
polyalkylene glycols.

The addition products of ethylene oxide and/or of propylene oxide with fatty alcohols, fatty acids, alkylphenols, glycerol mono- and di-esters and also sorbitan mono- and di-esters of fatty acids, or with castor oil, are known, commercially available products. They are usually homologue mixtures, the average degree of alkoxylation of which corresponds to the ratio of the amounts of ethylene oxide and/or propylene oxide and substrate with which the addition reaction is carried out. C₁₂-C₁₈ fatty acid mono- and di-esters of addition products of ethylene oxide with glycerol are known, for example, from DE-A-2 024 051 as fat-restoring substances for cosmetic preparations.

C₈-C₁₈Alkyl-mono- and -oligo-glycosides, their preparation and their use are known from the prior art. They are prepared especially by reacting glucose or oligosaccharides with primary alcohols having from 8 to 18 carbon atoms. Suitable glycoside radicals include mono-glycosides in which a cyclic sugar radical is glycosidically bonded to the fatty alcohol and also oligomeric glycosides having a degree of oligomerisation of up to preferably about 8. The degree of oligomerisation is a statistical average value based on a homologue distribution customary for such technical products.

It is also possible to use zwitterionic surfactants as emulsifiers. The term “zwitterionic surfactants” denotes especially surface-active compounds that carry at least one quaternary ammonium group and at least one carboxylate and/or sulfonate group in the molecule. Zwitterionic surfactants that are especially suitable are the so-called betaines, such as N-alkyl-N,N-dimethylammonium glycinates, for example cocoalkyldimethylammonium glycinate, N-acylaminopropyl-N,N-dimethylammonium glycinates, for example cocoacyl-aminopropyldimethylammonium glycinate, and 2-alkyl-3-carboxymethyl-3-hydroxy-ethylimidazolines each having from 8 to 18 carbon atoms in the alkyl or acyl group and also cocoacylaminoethylhydroxyethylcarboxymethyl glycinate. Special preference is given to the fatty acid amide derivative known by the CTFA name cocamidopropyl betaine. Likewise suitable as emulsifiers are ampholytic surfactants. Ampholytic surfactants are to be understood as meaning especially those which, in addition to containing a C₈-C₁₈-alkyl or -acyl group, contain at least one free amino group and at least one —COOH or —SO₃H group in the molecule and are capable of forming internal salts.

Examples of suitable ampholytic surfactants include N-alkylglycines, N-alkylpropionic acids, N-alkylaminobutyric acids, N-alkyliminodipropionic acids, N-hydroxyethyl-N-alkylamidopropyl-glycines, N-alkyltaurines, N-alkylsarcosines, 2-alkylaminopropionic acids and alkylaminoacetic acids, each having about from 8 to 18 carbon atoms in the alkyl group. Ampholytic surfactants to which special preference is given are N-cocoalkylaminopropionate, cocoacylaminoethylaminopropionate and C₁₂-C₁₈acylsarcosine. In addition to the ampholytic emulsifiers there also come into consideration quaternary emulsifiers, special preference being given to those of the esterquat type, preferably methyl-quaternised di-fatty acid triethanolamine ester salts.

Non-ionic emulsifiers are preferred. Of the non-ionic emulsifiers mentioned, special preference is given to ethoxylated fatty alcohols having from 8 to 22 carbon atoms and from 4 to 30 EO units.

The emulsifiers may be used in an amount of, for example, from 1 to 30% by weight, especially from 4 to 20% by weight and preferably from 5 to 10% by weight, based on the total weight of the composition. It is, however, also possible in principle to dispense with the use of emulsifiers.

The compositions according to the invention, for example creams, gels, lotions, alcoholic and aqueous/alcoholic solutions, emulsions, wax/fat compositions, stick preparations, powders or ointments, may in addition comprise, as further adjuvants and additives, mild surfactants, super-fatting agents, pearlescent waxes, consistency regulators, thickeners, polymers, silicone compounds, fats, waxes, stabilisers, biogenic active ingredients, deodorising active ingredients, anti-dandruff agents, film formers, swelling agents, further UV light-protective factors, antioxidants, hydrotropic agents, preservatives, insect repellents, solubilisers, perfume oils, colorants, bacteria-inhibiting agents and the like.

Substances suitable for use as super-fatting agents are, for example, lanolin and lecithin and also polyethoxylated or acrylated lanolin and lecithin derivatives, polyol fatty acid esters, monoglycerides and fatty acid alkanolamides, the latter simultaneously acting as foam stabilisers.

Examples of suitable mild surfactants, that is to say surfactants especially well tolerated by the skin, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, α-olefin sulfonates, ethercarboxylic acids, alkyl oligoglucosides, fatty acid glucamides, alkylamidobetaines and/or protein fatty acid condensation products, the latter preferably being based on wheat proteins.

As pearlescent waxes there come into consideration, for example: alkylene glycol esters, especially ethylene glycol distearate; fatty acid alkanolamides, especially coconut fatty acid diethanolamide; partial glycerides, especially stearic acid monoglyceride; esters of polyvalent, unsubstituted or hydroxy-substituted carboxylic acids with fatty alcohols having from 6 to 22 carbon atoms, especially long-chained esters of tartaric acid; fatty substances, for example fatty alcohols, fatty ketones, fatty aldehydes, fatty ethers and fatty carbonates, which in total have at least 24 carbon atoms, especially laurone and distearyl ether; fatty acids, such as stearic acid, hydroxystearic acid or behenic acid, ring-opening products of olefin epoxides having from 12 to 22 carbon atoms with fatty alcohols having from 12 to 22 carbon atoms and/or polyols having from 2 to 15 carbon atoms and from 2 to 10 hydroxy groups, and mixtures thereof.

As consistency regulators there come into consideration especially fatty alcohols or hydroxy fatty alcohols having from 12 to 22 carbon atoms and preferably from 16 to 18 carbon atoms, and in addition partial glycerides, fatty acids and hydroxy fatty acids. Preference is given to a combination of such substances with alkyl-oligoglucosides and/or fatty acid N-methylglucamides of identical chain length and/or polyglycerol poly-12-hydroxystearates. Suitable thickeners include, for example, Aerosil types (hydrophilic silicic acids), polysaccharides, especially xanthan gum, guar-guar, agar-agar, alginates and Tyloses, carboxymethyl cellulose and hydroxymethyl cellulose, also higher molecular weight poly-ethylene glycol mono- and di-esters of fatty acids, polyacrylates (e.g. Carbopols® from Goodrich or Synthalens® from Sigma), polyacrylamides, polyvinyl alcohol and polyvinyl-pyrrolidone, surfactants, for example ethoxylated fatty acid glycerides, esters of fatty acids with polyols, for example pentaerythritol or trimethylolpropane, fatty alcohol ethoxylates with restricted homologue distribution and alkyl-oligoglucosides as well as electrolytes, such as sodium chloride or ammonium chloride.

Suitable cationic polymers are, for example, cationic cellulose derivatives, for example a quaternised hydroxymethyl cellulose obtainable under the name Polymer JR 400® from Amerchol, cationic starches, copolymers of diallylammonium salts and acrylamides, quaternised vinylpyrrolidone/vinyl imidazole polymers, for example Luviquate (BASF), condensation products of polyglycols and amines, quarternised collagen polypeptides, for example lauryldimonium hydroxypropyl hydrolyzed collagen (Lamequat®L/Grünau), quaternised wheat polypeptides, polyethyleneimine, cationic silicone polymers, for example amidomethicones, copolymers of adipic acid and dimethylaminohydroxypropyldiethylene-triamine (Cartaretins®/Sandoz), copolymers of acrylic acid with dimethyldiallylammonium chloride (Merquat® 550/Chemviron), polyaminopolyamides, as described, for example, in FR-A-2 252 840, and the crosslinked water-soluble polymers thereof, cationic chitin derivatives, for example quaternised chitosan, optionally distributed in microcrystalline form; condensation products of dihaloalkyls, for example dibromobutane, with bisdialkylamines, for example bisdimethylamino-1,3-propane, cationic guar gum, for example Jaguar® C-17, Jaguar® C-16 from Celanese, quaternised ammonium salt polymers, for example Mirapol® A-15, Mirapol® AD-1, Mirapole AZ-1 from Miranol.

As anionic, zwitterionic, amphoteric and non-ionic polymers there come into consideration, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyltrimethylammonium chloride/acrylate copolymers, octyl acrylamide/methyl methacrylate/tert-butylaminoethyl methacrylate/2-hydroxypropyl methacrylate copolymers, polyvinylpyrrolidone, vinyl-pyrrolidone/vinyl acetate copolymers, vinylpyrrolidone/dimethylaminoethyl methacrylate/vinyl caprolactam terpolymers and also optionally derivatised cellulose ethers and silicones.

Suitable silicone compounds are, for example, dimethylpolysiloxanes, methylphenyl-polysiloxanes, cyclic silicones, and also amino-, fatty acid-, alcohol-, polyether-, epoxy-, fluorine-, glycoside- and/or alkyl-modified silicone compounds, which at room temperature may be in either liquid or resinous form. Also suitable are simethicones, which are mixtures of dimethicones having an average chain length of from 200 to 300 dimethylsiloxane units with hydrogenated silicates. A detailed survey by Todd et al. of suitable volatile silicones may also be found in Cosm. Toil. 91, 27 (1976).

Typical examples of fats are glycerides, and as waxes there come into consideration, inter alia, beeswax, carnauba wax, candelilla wax, montan wax, paraffin wax, hydrogenated castor oils, and fatty acid esters or microwaxes solid at room temperature optionally in combination with hydrophilic waxes, e.g. cetylstearyl alcohol or partial glycerides. Metal salts of fatty acids, for example magnesium, aluminium and/or zinc stearate or ricinoleate, may be used as stabilisers.

Biogenic active ingredients are to be understood as being, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudo-ceramides, essential oils, plant extracts, nucleic acids/bases, phosphorylated bases and oligonucleotides.

As deodorising active ingredients there come into consideration, for example, antiperspirants, for example aluminium chlorohydrates, which are colourless hygroscopic crystals that deliquesce readily in air and form aqueous aluminium chloride solutions when concentrated by evaporation. Aluminium chlorohydrate is used in the production of antiperspirant and deodorant preparations and presumably acts by partially closing the sweat glands as a result of protein and/or polysaccharide precipitation (see J. Soc. Cosm. Chem. 24, 281 (1973)). Under the trade mark Locron® of Hoechst AG, Frankfurt (FRG), there is available commercially, for example, an aluminium chlorohydrate corresponding to the formula Al₂(OH)₅Cl×2.5H₂O, the use of which is especially preferred (see J. Pharm. Pharmacol. 26, 531 (1975)). Besides the chlorohydrates it is also possible to use aluminium hydroxyacetates and acidic aluminium/zirconium salts. Esterase inhibitors may be added as further deodorising active ingredients. Such inhibitors are preferably trialkyl citrates, such as trimethyl citrate, tripropyl citrate, triisopropyl citrate, tributyl citrate and especially triethyl citrate (Hydagen® CAT, Henkel KGaA, Dusseldorf/FRG), which inhibit enzyme activity and hence reduce odour formation. It is likely that the cleavage of the citric acid ester releases the free acid which lowers the pH value on the skin to such an extent that the enzymes are inhibited. Further substances that come into consideration as esterase inhibitors are sterol sulfates or phosphates, for example lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester and hydroxycarboxylic acids and esters thereof, for example citric acid, malic acid, tartaric acid or tartaric acid diethyl ester. Antibacterial active ingredients that influence the germ flora and kill or inhibit the growth of sweat-decomposing bacteria can likewise be present in the preparations (especially in stick preparations). Examples thereof include chitosan, phenoxy-ethanol, chlorhexidine gluconate and 4-(2-tert-butyl-5-methylphenoxy)-phenol.

To improve the flow behaviour it is also possible to employ hydrotropic agents, for example ethanol, isopropyl alcohol or polyols. The polyols that come into consideration for that purpose have preferably from 2 to 15 carbon atoms and at least two hydroxy groups. The polyols may also contain further functional groups, especially amino groups, and/or may be modified with nitrogen. Typical examples are:
glycerol;
alkylene glycols, for example ethylene glycol, diethylene glycol, propylene glycol, butylene glycol, hexylene glycol and also polyethylene glycols having an average molecular weight of from 100 to 1000 Dalton;
technical oligoglycerol mixtures having an intrinsic degree of condensation of from 1.5 to 10, for example technical diglycerol mixtures having a diglycerol content of from 40 to 50% by weight;
methylol compounds, such as, especially, trimethylolethane, trimethylolpropane, trimethylolbutane, pentaerythritol and dipentaerythritol;
lower alkyl-glucosides, especially those having from 1 to 8 carbon atoms in the alkyl radical, for example methyl and butyl glucoside;
sugar alcohols having from 5 to 12 carbon atoms, for example sorbitol or mannitol;
sugars having from 5 to 12 carbon atoms, for example glucose or saccharose;
amino sugars, for example glucamine;
dialcohol amines, such as diethanolamine or 2-amino-1,3-propanediol.

Suitable preservatives include, for example, phenoxyethanol, formaldehyde solution, parabens, pentanediol or sorbic acid and the further substance classes listed in Appendix 6, Parts A and B of the Cosmetics Regulations. Suitable insect repellents include, for example, N,N-diethyl-m-toluamide, 1,2-pentanediol and insect repellent 3535.

There may be mentioned as perfume oils mixtures of natural and/or synthetic aromatic substances. Natural aromatic substances are, for example, extracts from blossom (lilies, lavender, roses, jasmine, neroli, ylang-ylang), from stems and leaves (geranium, patchouli, petitgrain), from fruit (aniseed, coriander, carraway, juniper), from fruit peel (bergamot, lemons, oranges), from roots (mace, angelica, celery, cardamom, costus, iris, calmus), from wood (pinewood, sandalwood, guaiacum wood, cedarwood, rosewood), from herbs and grasses (tarragon, lemon grass, sage, thyme), from needles and twigs (spruce, pine, Scots pine, mountain pine), and from resins and balsams (galbanum, elemi, benzoin, myrrh, olibanum, opoponax). Animal raw materials also come into consideration, for example civet and castoreum. Typical synthetic aromatic substances are, for example, products of the ester, ether, aldehyde, ketone, alcohol or hydrocarbon type. Aromatic substance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether; the aldehydes include, for example, the linear alkanals having from 8 to 18 hydrocarbon atoms, citral, citronellal, citronellyl oxyacetaldehyde, cyclamen aldehyde, hydroxycitronellal, lilial and bourgeonal; the ketones include, for example, the ionones, α,β-isomethylionone and methyl cedryl ketone; the alcohols include, for example, anethol, citronellol, eugenol, isoeugenol, geraniol, linalool, phenyl ethyl alcohol and terpineol; and the hydrocarbons include mainly the terpenes and balsams. It is preferable, however, to use mixtures of different aromatic substances that together produce an attractive scent. Ethereal oils of relatively low volatility, which are chiefly used as aroma components, are also suitable as perfume oils, e.g. sage oil, camomile oil, clove oil, melissa oil, oil of cinnamon leaves, lime blossom oil, juniper berry oil, vetiver oil, olibanum oil, galbanum oil, labolanum oil and lavandin oil. Preference is given to the use of bergamot oil, dihydromyrcenol, lilial, lyral, citronellol, phenyl ethyl alcohol, geraniol, benzyl acetone, cyclamen aldehyde, linalool, boisambrene forte, ambroxan, indole, hedione, sandelice, lemon oil, tangerine oil, orange oil, allyl amyl glycolate, cyclovertal, lavandin oil, muscatel sage oil, α-damascone, bourbon geranium oil, cyclohexyl salicylate, vertofix coeur, iso-E-Super, Fixolide NP, evernyl, iraldein gamma, phenylacetic acid, geranyl acetate, benzyl acetate, rose oxide, romillat, irotyl and floramat alone or in admixture with one another.

There may be used as colorants the substances that are suitable and permitted for cosmetic purposes, as compiled, for example, in the publication “Kosmetische Farbemittel” of the Farbstoffkommission der Deutschen Forschungsgemeinschaft, Verlag Chemie, Weinheim, 1984, pages 81 to 106. The colorants are usually used in concentrations of from 0.001 to 0.1% by weight, based on the total mixture.

Typical examples of bacteria-inhibiting agents are preservatives that have a specific action against gram-positive bacteria, such as chlorhexidine (1,6-di(4-chlorophenyl-biguanido)hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorising agent of interest is the terpene alcohol farnesol (3,7,11-trimethyl-2,6,10-dodecatrien-1-ol), which is present in lime blossom oil and also fractions from the steam extract of the wood and/or bark of Callistris intratropica, which is also known as “blue cypress oil”. Glycerol monolaurate has also proved to be a bacteriostatic agent. The amount of the additional bacteria-inhibiting agents present is usually from 0.1 to 2% by weight, based on the solids content of the preparations.

It is furthermore possible for the cosmetic compositions to comprise as adjuvants anti-foams, such as silicones, structurants, such as maleic acid, solubilisers, such as ethylene glycol, propylene glycol, glycerol or diethylene glycol, opacifiers, such as latex, styrene/PVP or styrene/acrylamide copolymers, complexing agents, such as EDTA, NTA, β-alaninediacetic acid or phosphonic acids, propellants, such as propane/butane mixtures, N₂O, dimethyl ether, CO₂, N₂ or air, so-called coupler and developer components as oxidation dye precursors, reducing agents, such as thioglycolic acid and derivatives thereof, thiolactic acid, cysteamine, thiomalic acid or α-mercaptoethanesulfonic acid, or oxidising agents, such as hydrogen peroxide, potassium bromate or sodium bromate.

Cosmetic formulations according to the invention are contained in a wide variety of cosmetic preparations. There come into consideration, for example, especially the following preparations:
skin treatment preparations for the purpose of lightening, depigmenting or bleaching the skin;
preparations for removing freckles, age spots or liver spots or marks or for treating hyperpigmentation;
skin-care preparations, e.g. skin-washing and cleansing preparations in the form of tablet-form or liquid soaps, synthetic detergents or washing pastes;
skin-care preparations, e.g. body lotions, skin emulsions, multi-emulsions or skin oils;
cosmetic personal care preparations, e.g. facial make-up in the form of day creams or powder creams, face powder (loose or pressed), rouge or cream make-up, lip-care preparations, e.g. lipsticks, lip gloss, lip contour pencils, nail-care preparations, such as nail varnish, nail varnish removers, nail hardeners or cuticle removers;
special deodorants and antiperspirants or callus-removing preparations;
light-protective preparations, such as sun milks, lotions, creams or oils, sunblocks or tropicals, or after-sun preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, shaving foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams.

The final formulations listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, cream, milk or lotion,
in the form of make-up,
in the form of a stick,
in the form of a spray (spray with propellent gas or pump-action spray) or an aerosol,
in the form of a foam,
in the form of a paste, or
in the form of surfactant-containing cleansing preparations.

Of special importance as cosmetic compositions for the skin are:
skin treatment preparations for the purpose of lightening, depigmenting or bleaching the skin; preparations for removing freckles, age spots or liver spots or marks or for treating hyperpigmentation, such as skin milks, lotions, creams, oils, gels and sprays as well as pastes or soaps, such as solid or liquid soaps;
light-protective preparations, such as sun milks, lotions, creams, oils, sunblocks or tropicals, after-sun preparations. Of particular interest are sun protection creams, sun protection lotions, sun protection oils, sun protection milk and sun protection preparations in the form of a spray.

When the compositions according to the invention are in the form of a cleansing preparation, they comprise at least one cleansing substance. Suitable cleansing substances are, for example, anionic, non-ionic or zwitterionic and amphoteric synthetic, cleansing substances.

Suitable anionic cleansing substances are
sulfates, e.g. fatty alcohol sulfates having an alkyl chain length of from 8 to 18 carbon atoms, for example sulfated lauryl alcohol;
fatty alcohol ether sulfates, e.g. the acidic esters or salts thereof of a polyadduct of from 2 to 30 mol of ethylene oxide with 1 mol of a C₈-C₂₂ fatty alcohol;
the alkali metal, ammonium or amine salts of C₈-C₂₀ fatty acids that are designated soaps, e.g. coconut fatty acid;
alkylamide sulfates;
alkylamine sulfates, e.g. monoethanolamine lauryl sulfate;
alkylamide ether sulfates;
alkylaryl polyether sulfates;
monoglyceride sulfates;
alkanesulfonates having an alkyl chain length of from 8 to 20 carbon atoms, e.g. dodecyl sulfonate;
alkylamide sulfonates;
alkylarylsulfonates;
α-olefin sulfonates;
sulfosuccinic acid derivatives, e.g. alkylsulfosuccinates, alkyl ether sulfosuccinates or alkylsulfosuccinamide derivatives;
   N-(alkylamidoalkyl)amino acids of formula

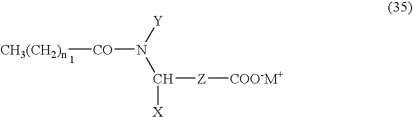

wherein
x is hydrogen; C₁-C₄alkyl or —COOM⁺;
Y is hydrogen or C₁-C₄alkyl;
Z is —(CH₂)ₘ₁₋₁;
m₁ is an integer from 1 to 5;
n1 is an integer from 6 to 18 and
M is an alkali metal or ammonium cation;
   alkyl and alkaryl ether carboxylates of formula

CH₃—X—Y-A (34)
wherein
X is a radical

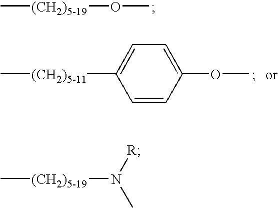

R is hydrogen; or C₁-C₄alkyl;
Y is —(CHCHO)₁₋₅₀—;
A is —(CH₂)ₘ₂₋₁COO⁻M⁺; or

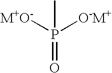

m₂ is from 1 to 6 and
M is an alkali metal or amine cation.

Also used as anionic surfactants are fatty acid methyl taurides, alkyl isothionates, fatty acid polypeptide condensation products and fatty alcohol phosphoric acid esters. The alkyl radicals occurring in those compounds preferably have from 8 to 24 carbon atoms.

The anionic surfactants are generally in the form of their water-soluble salts, such as the alkali metal, ammonium or amine salts. Examples of such salts are lithium, sodium, potassium, ammonium, triethylamine, ethanolamine, diethanolamine and triethanolamine salts. There are especially used the sodium, potassium or ammonium (NR₁R₂R₃) salts wherein R₁, R₂ and R₃ are each independently of the others hydrogen, C₁-C₄alkyl or C₁-C₄hydroxyalkyl.

Very especially preferred anionic surfactants in the composition according to the invention are monoethanolamine lauryl sulfate or the alkali metal salts of fatty alcohol sulfates, especially sodium lauryl sulfate and the reaction product of from 2 to 4 mol of ethylene oxide and sodium lauryl ether sulfate.

As zwitterionic and amphoteric surfactants there come into consideration C₈-C₁₈betaines, C₈-C₁₈sulfobetaines, C₈-C₂₄alkylamido-C₁-C₄alkylenebetaines, imidazoline carboxylates, alkylamphocarboxycarboxylic acids, alkylamphocarboxylic acids (e.g. lauroamphoglycinate) and N-alkyl-b-amino-propionates or -iminodipropionates, preference being given to the C₁₀-C₂₀alkylamido-C₁-C₄alkylenebetaines and especially coconut fatty acid amide propylbetaine.

As non-ionic surfactants there are suitable, for example, derivatives of the adducts of propylene oxide/ethylene oxide with a molecular weight of from 1000 to 15 000, fatty alcohol ethoxylates (1-50 EO), alkylphenol polyglycol ethers (1-50 EO), ethoxylated carbohydrates, fatty acid glycol partial esters, for example diethylene glycol monostearate, fatty acid alkanol amides and dialkanol amides, fatty acid alkanol amide ethoxylates and fatty amine oxides.

It is also possible to use the salts of saturated and unsaturated C₈-C₂₂ fatty acids either alone, in admixture with one another or in admixture with the other cleansing substances mentioned above. Examples of those fatty acids are capric, lauric, myristic, palmitic, stearic, arachic, behenic, caproleic, dodecenoic, tetradecenoic, octadecenoic, oleic, eicosenoic and erucic acids, and the technical mixtures of those acids, e.g. coconut fatty acid. Such acids are in the form of salts, suitable cations being alkali metal cations, such as sodium and potassium cations, metal atoms, such as zinc and aluminium atoms, or sufficiently alkaline-reacting, nitrogen-containing, organic compounds such as amines or ethoxylated amines. Such salts can also be prepared in situ.

In the composition according to the invention there is preferably used a soap, that is to say a branched or unbranched long-chained alkyl- or alkenyl-carboxylic acid salt, e.g. a sodium, potassium, ammonium or substituted ammonium salt.

The cleansing composition according to the invention may be in solid form, in the form of a gel, a synthetic detergent or a liquid formulation. It can be prepared in accordance with customary methods.

The soaps (solid soaps, synthetic detergents, liquid soaps) are prepared in accordance with procedures customary for such products generally in the soap industry and described in the literature (see e.g. L. Spitz (Ed.), Soaps and Detergents, A Theoretical and Practical Review, AOCS Press, Champaign, Ill., USA (1996)). A crucial factor in the production of solid soaps is intensive mixing of the soap mass before extrusion, in order that homogeneous distribution of the ingredients, especially the antioxidant, is achieved. The antioxidant is usually added to the soap mass directly or, if desired, predissolved in perfume, homogeneously distributed therein by mixing (for example in a jetstream mixer) and kneading (for example in an intensive kneader) before the mass is extruded or compressed in a mould. Liquid soaps are likewise produced by homogenisation of the constituents in suitable mixing apparatus (e.g. Sulzer mixers, Erestat mixers or DAT mixers from Pfaudler), the uniform distribution of the antioxidant generally being achieved more quickly than in the case of solid soaps on account of the lower viscosity of the formulation. As a departure from that procedure, it is also possible for the antioxidant to have been incorporated into the soap base (flocks, noodles), optionally with the application of heat (melt-incorporation).

The following Examples serve to illustrate the invention, but do not limit the invention thereto.

### EXAMPLES

### Formulation Examples

### Example 1

### Basic Formulation

| | % by | Compo- | |
|---|---|---|---|
| | weight | nent | Chemical name |
| Part A: | 4.5 | A₁ | glycyl stearate |
| | 5.00 | A₂ | hydrogenated polydecene |
| Part B: | qs 100 | B | deionised water |
| Part C: | 0.10 | C₁ | 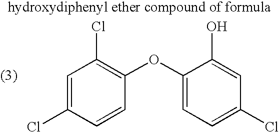 |
| | 5.0 | C₂ | propylene glycol |
| Part D: | 0.02 | D₁ | caramel color |
| | 0.02 | D₂ | NaOH |
| | 0.60 | D₃ | phenoxyethanol/methylparaben/ethylparaben/butyl- |
| | | | paraben/propylparaben/isobutylparaben (Phenonip) |
| | 0.80 | D₄ | Polyquaternium-37/propylene glycol dicaprylate dicaptrate, |
| | | | PPG-1/Trideceth-6 |

Preparation: Part A is heated to 75° C. C₁ is dispersed in C₂.

Part B is heated to 75° C. Before emulsification, Part C is added to Part A. Part A is added to Part B with cautious stirring. Homogenisation is then carried out for 10 seconds using an Ultra Turrax.

The mixture is then allowed to cool to 60° C., with cautious stirring, and D₂ is added to the emulsion. D₃ is added, with stirring. Further stirring is carried out until a temperature of 40° C. is reached. D₁ is added and the pH is adjusted to 6.7.

### Example 2

### Face Cream Formulation

A face cream is prepared and contains the following constituents:

3.0% by weight glycerol monostearate,
1.5% by weight beeswax,
0.5% by weight sorbitan monooleate,
5.0% by weight Vaseline, liq.
10.0% by weight paraffin,
1.0% by weight lecithin,
0.5% by weight sodium N-stearoyl-L-glutamate,
0.15% by weight xanthan gum,
0.2% by weight compound of formula (3),
0.1% by weight kojic acid,
0.1% by weight methylparaben, and
H₂O ad 100% by weight.

### Example 3

### Spray Formulation

0.1-0.5% by weight compound of formula (3)
0.5-0.1% by weight kojic acid;
2.0% by weight Solubilisant LRI (LCW),
70% by weight ethanol_{abs}
water ad 100%.

### Example 4

### Oil-in-Water Formulation

0.05-0.5% by weight compound of formula (3) and a different skin-lightening substance;
0.05-5% by weight one or more UV absorbers;
12% by weight glyceryl stearate;
6% by weight paraffin oil;
6% by weight caprylic/capric triglyceride;
4% by weight glycerol;
0.2% by weight disodium EDTA
1.0% by weight citrate and
water to 100 parts by weight.

### Example 5

### Tablet Soap Formulation

| | Active soap |
|---|---|
| INCI name | % (as supplied) |
| hydroxydiphenyl ether compound of formula (3) | 0.5 |
| titanium dioxide | 0.2 |
| tetrasodium EDTA | 0.023 |
| stearic acid | 3.0 |
| glycerol-water | qs |
| Vibracolor Green PGR7-I | 0.004 |
| Vibracolor Red PRE5-L | — |
| soap noodles | ad 100 |

### Example 6

### Half-Side Comparison with Soar Formulations

Groups of 20 Asian subjects (China, Indochina, collectively having L* and b* values within 8 L* and b* values, respectively) cleansed the test areas twice daily for 30-40 sec each time with soaps a) and b) and with soaps c) and d), rinsing off again after 30 sec.

Before the first treatment and at 4, 6, 8 and 12 weeks, the colour values of a treatment group on the test areas on the forearms and on the lower legs were measured and averaged. The soaps were used in a blind test; they were allocated randomly to the left or right side.

The following soap composition w were used:

a) soap containing 0.3% compound of formula (3)
b) control soap containing (without active ingredients)
c) control soap containing 0.3% of compound of formula (FW-22)
d) soap with Immediate White (0.3% of compound of formula (FW-22) and 0.3% of the compound of formula (3).

The results of the measurements are shown in Table 6.

TABLE 6

| Colour L*a*b* | — | start | 4 weeks | 6 weeks | 8 weeks | 12 weeks |
|---|---|---|---|---|---|---|
| a) | L* | 54.8 | 55.1; | 58.6 | 60.0 | 63.3 |
| | a* | 19.9 | 20.2; | 21.5 | 22.9 | 22.7 |
| | b* | 19.3 | 18.9 | 18.3 | 17.9 | 18.4 |
| b) | L* | 55.6 | 55.6 | 55.4 | 55.6 | 54.3 |
| | a* | 19.9 | 19.9 | 21.8 | 23.1 | 22.7 |
| | b* | 19.5 | 19.5 | 19.2 | 19.1 | 19.9 |
| c) | L* | 53.4 | 54.9; | 53.9 | 55.0 | 54.4 |
| | a* | 19.7 | 20.0; | 21.4 | 22.8 | 21.9 |
| | b* | 19.5 | 18.7 | 18.1 | 18.0 | 18.7 |
| d) | L* | 55.5/58.4 | 59.5 | 62.9 | 64.1 | 67.8 |
| | a* | 19.8/19.4 | 21.1 | 22.2 | 22.9 | 23.2 |
| | b* | 19.3/19.7 | 20.3 | 21.1 | 21.6 | 21.8 |

### Formulation:

X % mixture comprising

| | | |
|---|---|---|
| compound of formula (3) | 10 | parts |
| compound of formula (FW-22) | 10 | parts |
| compound of formula (AO 26) | 1 | part |

is stirred into soap noodles and then the remaining substances are added.

| Ingredients | (a) | (b) | (d) |
|---|---|---|---|
| titanium oxide | 0.2 | 0.2 | 0.2 |
| tetrasodium EDTA | 0.023 | 0.023 | 0.023 |
| glycerol-water | qs | qs | qs |
| stearic acid | 3.0 | 3.0 | 3.0 |
| Vibracolor Green PGR7-1 | 0.004 | — | — |
| Vibracolor Red PRE5-L | — | — | 0.004 |
| hydroxydiphenyl ether | 0.3 | | |
| compound | | | |
| Immediate White | | | 0.63 |
| antioxidant (Tinogard TT) | 0.02 | 0.02 | |
| soap noodles | ad 100 | ad 100 | ad 100 |

What is claimed is:

1. A method of inhibiting melanogenesis and for lightening skin, which comprises contacting said skin with a composition comprising
from 0.001 to 10% based on weight of said composition of component (a) which is a halogenated hydroxydiphenyl ether compound selected from the group consisting of 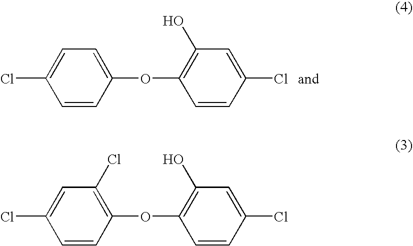
from 0.01 to 2% based on weight of said composition of component (b) which is a skin-lightening substance selected from the group consisting of arbutin, quercitin, aloesin, azelaic acid, guaiol, ellagic acid and ester compounds thereof and fluorescent whiteners; and
from 0.1 to 15% based on weight of said composition of component (c) one or more UV-A and/or UV-B absorbers selected from the group consisting of 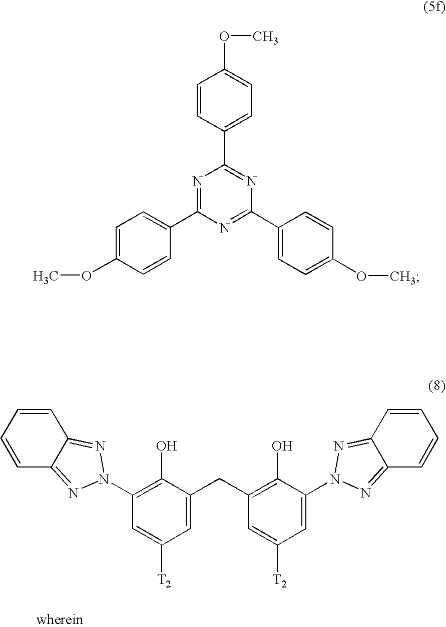
T₂ is C₁-C₁₂alkyl;
octyl methoxycinnamate; and benzophenone-3.

2. A method according to claim 1, wherein the composition comprises as component (c) the compound of formula

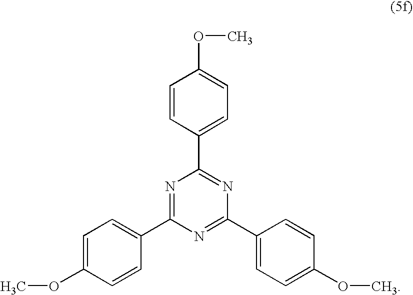

3. A method according to claim 1, wherein the composition comprises as component (c) the compound of formula 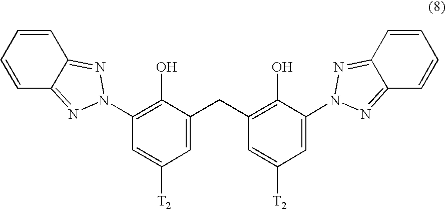
wherein
is C₁-C₁₂alkyl.

4. A method according to claim 3, wherein
T₂ is iso-octyl.

5. A method according to claim 1, wherein the composition comprises octyl methoxycinnamate as component (c).

6. A method according to claim 1, wherein the composition comprises benzophenone-3 as component (c).

7. A method wherein a composition according to claim 1 is incorporated into cosmetic formulations.

8. A method according to claim 7, wherein the composition is used in the form of an oil-in-water formulation, in a solvent formulation or in the form of a paste formulation.

9. A method according to claim 7, wherein the proportion of the composition in the formulation is from 0.01% to 10% parts by weight.

10. A cosmetic formulation, comprising
(a) a halogenated hydroxydiphenyl ether compound of formula 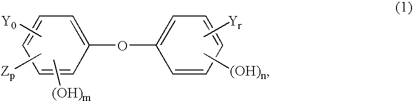
wherein
Y is chlorine or bromine,
Z is SO₂H, NO₂; or C₁-C₄alkyl;
m is 0 or 1;
n is 1 or 2;
r is from o to 3;
o is from 1 to 3; and
p is 0, 1 or 2;
and one or more of components (b) and/or (c):
wherein
(b) is a further skin-lightening active ingredient selected from the group consisting of pyrone derivatives, hydroquinone, hydroquinone glycosides, hydroquinone derivatives, resorcinol derivatives, glycine, glutathione, acetylcysteine, oligopeptides, alkyldicarboxylic acids, 1,2-dihydroxyphenyl derivatives, urea, allantoin, furanones, phenylacetaldehydes, benzaldehydes, 4-methoxycinnamaldehydes, isomeric decenoic acid, ascorbic acid and derivatives thereof, salicylic acid derivatives, phenolic substances, benzo[b]pyran derivatives, bornyl and cinnamate derivatives, azulene and derivatives thereof, cell messenger substances, fluorescent whiteners, and compounds of formulae 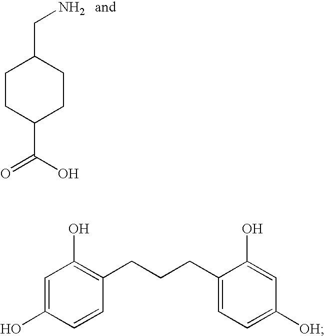
(c) is one or more UV-A and/or UV-B absorbers; and optionally
(d) is an antioxidant;
and also cosmetically acceptable adjuvants or carriers.

11. A cosmetic formulation according to claim 10, containing
from 0.001 to 10% by weight, based on the weight of total formulation, of component (a),
from 0 to 10% by weight, based on the weight of total formulation, of component (b),
from 0 to 30% by weight, based on the weight of total formulation, of component (c), and
from 0 to 30% by weight, based on the weight of total formulation, of component (d).

12. A cosmetic formulation according to claim 10, containing
from 0.05 to 2% of component (a), selected from the group consisting of 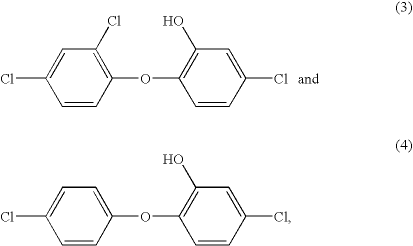
from 0.01 to 2% of component (b), selected from the compound of formula 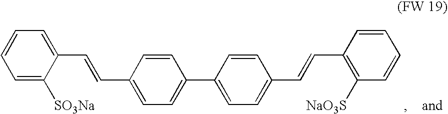
from 0.05 to 2 of % component (d), selected from the group consisting of

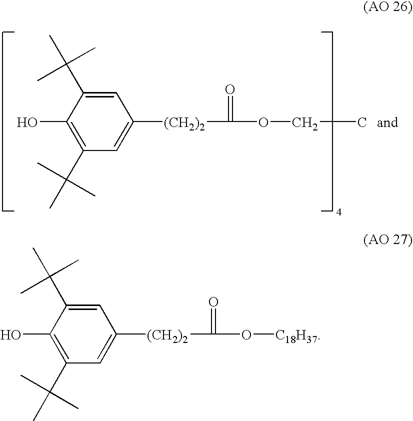

13. A method of inhibiting melanogenesis and for lightening skin, which comprises contacting said skin with an effective amount of a formulation according to claim 12 in surfactant-containing cleansing compositions.

14. Cleansing composition, comprising
0.05 to 2%, based on weight of the composition, of component (a),
0.001 to 2%, based on weight of the composition, of component (b),
0 to 2%, based on weight of the composition, of component (c),
0 to 2%, based on weight of the composition, of component (d), and
0.1 to 10%, based on weight of the composition, of one or more synthetic detergents or soaps or a combination of such substances, where components (a), (b), (c) and (d) are as defined in claim 10.

15. A cosmetic formulation according to claim 10, wherein component (b) is selected from the group consisting of kojic acid, alpha-arbutin, quercitin, aloesin, azelaic acid, guaiol, ellagic acid and esters thereof and also the fluorescent whitener of formula [chemical structure]
